(19) United States
(12) Patent Application Publication
(10) Pub. No.: US 2011/0166035 A1
(43) Pub. Date: Jul. 7, 2011

(54) NOVEL DIAGNOSTIC METHOD

(75) Inventors: Martin Kleinschmidt, Halle/Saale (DE); Claudia Goettlich, Halle/Saale (DE); Hans-Ulrich Demuth, Halle/Saale (DE); Jens-Ulrich Rahfeld, Ot Roeblingen am See (DE)

(73) Assignee: PROBIODRUG AG, Halle/Saale (DE)

(21) Appl. No.: 12/954,225

(22) Filed: Nov. 24, 2010

Related U.S. Application Data
(60) Provisional application No. 61/263,861, filed on Nov. 24, 2009.

(51) Int. Cl. C40B 30/04; G01N 33/53

(52) U.S. Cl. 506/9; 436/501; 435/7.94

(57) ABSTRACT

Provided herein are methods, compositions, and kits related to the detection and diagnosis of a neurodegenerative disorder, such as Alzheimer's disease. Various aspects use the oligomeric state of fragments of amyloid β as a biomarker and further concerns a novel method to determine the oligomeric state of fragments of amyloid β in biological samples.

### CROSS-REFERENCE TO RELATED APPLICATIONS

This application is the non-provisional of and claims priority to U.S. Provisional Application Ser. No. 61/263,861, filed on Nov. 24, 2009, which is incorporated herein by reference in its entirety.

### FIELD OF THE INVENTION

The invention relates to the detection and diagnosis of Alzheimer's disease with the use of the oligomeric state of fragments of amyloid β as a biomarker and further concerns a novel method to determine the oligomeric state of fragments of amyloid β in biological samples.

### BACKGROUND OF THE INVENTION

Alzheimer's disease is the most common form of dementia and has a prevalence of approximately 65-70% among all dementia disorders (Blennow et al., 2006). Resulting from increased life expectancy, this disease has become a particular issue in highly developed industrialised countries like Japan and China as well as in the US and Europe. The number of Alzheimer patients is estimated to increase from 24 million in 2001 to 81 million in 2040 (Ferri et al., 2005). Currently, the costs for treatment and care of AD patients worldwide amount to approximately 250 billion US dollars per year.

The progression of the sporadic form of the disease is relatively slow and Alzheimer's disease will usually last for about 10-12 years after the onset of first symptoms. Presently, it is extremely difficult to make a reliable and early diagnosis of AD and distinguish it from other forms of dementia. A good diagnosis with a reliability of more than 90% is only possible in the later stages of the disease. Prior to that, it is only possible to make a prediction that Alzheimer's is possible or probable; diagnosis here relies on the use of certain criteria according to Knopman et al., 2001; Waldemar et al., 2007 or Dubois et al., 2007. Neurodegeneration starts however 20 to 30 years before the first clinical symptoms are noticed (Blennow et al., 2006; Jellinger K A, 2007). The onset of the clinical phase is usually characterized by the so-called “mild cognitive impairment” (MCI), where patients will show measurable cognitive deficits which are not sufficient to enable a diagnosis of a dementia disease in a clear fashion (Petersen et al., 1999; Chetkow et al., 2008). Many patients with MCI will have neuropathological changes which are typical for AD and which means that an earlier stage of AD is possible, but not certain (Scheff et al., 2006; Markesbery et al., 2006; Bouwman et al., 2007). There are however many MCI cases which will not progress to Alzheimer's; in these cases, other factors are responsible for the cognitive deficit (Saito et al., 2007; Jicha et al., 2006 and Petersen et al., 2006). While some MCI patients will not show any deterioration of their condition or even some kind of amelioration, for most MCI cases the cognitive deficit will continue to clinical dementia. The yearly rate of this conversion is approximately 10-19% (Gauthier et al., 2006; Fischer et al., 2007). At present there is a combination of clinical, neuropsychological and imaging processes which are capable of differentiating the various subtypes of Mild Cognitive Impairment (Devanand et al., 2007; Rossi et al., 2007; Whitwell et al., 2007; Panza et al., 2007). However, there is no significant difference between these subtypes in relation to the further progression of dementia (Fischer et al., 2007). Thus, it is of utmost importance to develop a method to enable a clear and reliable diagnosis of Alzheimer's disease in the early stages, suitably at its onset or during MCI.

### Biomarkers

Biomarkers for Alzheimer's disease have already been described in the prior art. Alongside well known psychological tests such as e.g. ADAS-cog, MMSE, DemTect, SKT or the Clock Drawing test, biomarkers are supposed to improve diagnostic sensitivity and specificity for first diagnosis as well as for monitoring the progression of the disease. In relation to the current status of development of biomarkers for AD/MCI it was proposed to correlate the disease in the future with the other diagnostic criteria (Whitwell et al., 2007; Panza et al., 2007; Hyman SE, 2007). Biomarkers are supposed to support the classical neuro-psychological tests in the future. There is a common belief that they will be of great importance as surrogate markers for the development of agents against Alzheimer's (Blennow K, 2004; Blennow K, 2005; Hampel et al., 2006; Lewczuk et al., 2006; Irizarry M C, 2004).

### Structural Biomarkers

“Magnetic resonance imaging” (MRI) is an imaging process which allows detection of degenerative atrophies in the brain (Barnes et al., 2007; Vemuri et al., 2008). Thus, atrophy of the medial temporal lobe (MTA) is sensitive to a degeneration of the hippocampal region in the brain of older patients; this can be made visible very clearly by MRI, but is not specific for Alzheimer's disease. Mild MTA is not encountered more frequently in other dementias (Barkhof et al., 2007) but it does correlate with MCI (Mevel et al., 2007). For this reason it is not possible to determine from MRI data alone whether the neurodegeneration is Alzheimer's disease or an early stage of Alzheimer's disease. A further imaging method is Positron Emission Tomography (PET) which visualises the accumulation of a detector molecule (PIB) on amyloid deposits. It could be detected that the thioflavin T-analogue (¹¹C)PIB will accumulate increasingly in certain regions of the brain of patients with MCI or mild Alzheimer's disease, respectively (Kemppainen et al., 2007; Klunk et al., 2004; Rowe et al., 2007); unfortunately this can also be detected in subjects who do not have dementia (Pike et al., 2007). This would probably indicate that the detection of amyloid deposits via PET allows detection of pre-clinical stages of Alzheimer's; however, this has to be confirmed by further studies. Besides the most frequently used processes, MRI and PET, there are additional structural biomarkers for AD: CBF-SPECT, CMRg1-PET (glucose metabolism proton spectroscopy (H-1 MRS), high field strength functional MRI, voxel-based morphometry, enhanced activation of the mediobasal temporal lobe (detected by fMRI, (R)-[(¹¹)C]PK11195 PET for the detection of microglial cells (Huang et al., 2007; Kantarci et al., 2007; Petrella et al., 2007; Hamalainen et al., 2007; Kircher et al., 2007; Kropholler et al., 2007).

### CSF Biomarkers

Senile plaques are one of the pathological characteristics of Alzheimer's disease. These plaques consist mostly of Aβ (1-42) peptides (Attems J, 2005). In some studies it could be shown that a low level of Aβ (1-42) in CSF of MCI patients correlates specifically with the further development of Alzheimer's disease in its progression (Blennow and Hampel, 2003; Hansson et al., 2006 and 2007). The reduction in CSF is probably due to enhanced aggregation of Aβ (1-42) in the brain (Fagan et al., 2006; Prince et al., 2004; Strozyk et al., 2003). Another possibility is the occurrence of semi-soluble Aβ (1-42) oligomers (Walsh et al., 2005) which would lead to a lower level of detection in CSF. In particular in the early stages of Alzheimer's, decreased concentrations of Aβ (1-42) would be detected, while increased amounts of Tau protein and phospho-tau proteins in CSF, respectively, could be detected (Ewers et al., 2007; Lewczuk et al., 2004). To provide a better predictability of biomarkers, it is usually attempted to use the Tau/Aβ (1-42) ratio and correlate it with the prediction of cognitive deficiency in older persons who do not have dementia (Fagan et al., 2007; Gustafson et al., 2007; Hansson et al., 2007; Li et al., 2007; Stomrud et al., 2007) as well as in MCI patients (Hampel et al., 2004; Maccioni et al., 2006; Schönknecht et al., 2007). A further correlation between ante mortem CSF level of Aβ (1-42), Tau, phospho-Tau-Thr231 and post-mortem histopathological alterations of the brain could be detected in AD patients (Clark et al., 2003; Buerger et al., 2006). In other studies, however, no correlation between CSF biomarkers and Aβ (1-42), total Tau and phospho-Tau with APOE ε4-allele, plaque and tangle load after autopsy could be detected (Engelborghs et al., 2007; Buerger et al., 2007). An interesting aspect was detected in a multicenter study. It appears that increased level of total Tau and phospho-Tau (181) correlates with a decreased ratio of Aβ (1-42)/Aβ (1-40), but not with the Aβ (1-42) alone (Wiltfang et al., 2007). An increased level of CSF Tau was however also detected in other CNS diseases such as Creutzfeldt-Jakob disease, brain infarction, and cerebral vascular dementia, which are all associated with a neuronal loss (Buerger et al., 2006 (2); Bibl et al., 2008). A further possible biomarker is the increase of BACE 1 activity in CSF as an indicator for MCI (Zhong et al., 2007). It is also discussed that the increased BACE 1 activity will result in increased Aβ production and therefore increased aggregation of the peptides. Alzheimer's disease is accompanied by neuroinflammatory processes. CSF anti-microglial cell antibodies are therefore possible biomarkers for these inflammatory processes in AD (McRea et al., 2007).

In spite of the multitude of biomarkers which are supposed to enable early diagnosis of Alzheimer's disease, there is not a single biomarker that ensures reliable and clear diagnosis. This is usually because most studies use a comparison of the respective biomarkers and clinical diagnosis. A better approach would be the correlation of biomarkers with the pathological causes of Alzheimer's disease.

A possible approach would be repeated analysis of immuno-precipitated CSF samples of clearly identified and defined neuropathological dementia diseases to clarify whether Aβ (1-40) and Aβ (1-42) are in fact suitable neurochemical dementia markers (Jellinger et al., 2008). In order to discover novel, up to now unknown, biomarkers for Alzheimer's disease, CSF samples are usually analyzed via a comparative proteomic analysis which results in a diagnosis of AD with enhanced sensitivity and also to enable the differentiation from other degenerative dementia disorders (Finehout et al., 2007; Castano et al., 2006; Zhang et al., 2005; Simonsen et al., 2007; Lescuyer et al., 2004; Abdi et al., 2006). After a proteomic analysis, the potential new biomarker should be analyzed in detail for its suitability and correlation with pathological causes. A typical example for a biomarker which was found by a proteomic analysis is truncated cystatin C as a biomarker for multiple sclerosis; this biomarker was later proven to be a storage artefact (Irani et al., 2006; Hansson et al., 2007 (2)).

### Plasma Biomarkers

Besides the frequently used plasma biomarkers, i.e. the Aβ peptides, further inflammatory plasma markers are used for the early diagnosis of dementia (Ravaglia et al., 2007; Engelhart et al., 2004) in particular for Alzheimer's (Motta et al., 2007). All of these are still under discussion. Further possible biomarkers were also found via comparative proteomic analysis of plasma from AD patients and healthy controls (German et al., 2007; Ray et al., 2007). The future will show whether these biomolecules are indeed specific for Alzheimer's disease and are suitable as biomarkers. There is no convincing or suitable data which would show either specificity or suitability of any of the biomarkers discussed above.

Contrary to the analysis of amyloid p in CSF, the results until now with respect to suitable Aβ biomarkers in plasma are not reliable or clear. In some studies a correlation between a decreased ratio of Aβ (1-42)/Aβ (1-40) in plasma and an enhanced conversion of cognitive normal persons to MCI or Alzheimer patients, respectively, was found ((Graff-Radford et al., 2007; van Oijen et al., 2006; Sundelof et al., 2008). Other studies however detected that a reduction of the Aβ (1-42) plasma level is more likely a marker for the conversion from MCI to AD (Song et al., 2007) and is not suitable as a marker for neurodegenerative purposes which are encountered with Alzheimer's (Pesaresi et al., 2006). Most of the studies however do not show a difference in Aβ plasma levels between healthy controls and patients with sporadic Alzheimer's (Fukumoto et al., 2003; Kosaka et al., 1997; Scheuner et al., 1996; Sobow et al., 2005; Tamaoka et al., 1996; Vanderstichele et al., 2000). Some studies also showed that the level of Aβ in plasma does not correlate with the level as encountered in the brain (Fagan et al., 2006; Freeman et al., 2007) nor does it correlate with the level encountered in CSF (Mehta et al., 2001; Vanderstichele et al., 2000). In a recent study, a correlation was detected for Aβ (1-40) and Aβ (1-42) between CSF and plasma, but only in healthy controls. This correlation could not be detected in MCI and AD which is explained by destroying the balance between CSF and plasma Aβ due to Aβ deposits in the brain (Giedraitis et al., 2007). Generally, it is assumed that plasma Aβ (1-42) level is not a reliable biomarker for MCI or AD (Blasko et al., 2008; Mehta et al., 2000; Brettschneider et al., 2005), whereas a decrease of the ratio plasma Aβ (1-38)/Aβ (1-40) is considered a biomarker for vascular dementia and comes close to the predictability of CSF markers (Bibl et al., 2007).

Until now, Aβ oligomers were disregarded as biomarkers for Alzheimer, however, they are supposed to play a decisive role in initiating the neurodegenerative process (Walsh & Selkoe, 2007). In several studies, the neurotoxic effect was shown for Aβ dimers with 8 kDa to the point of protofibrils with over 100 kDa (Lambert et al., 1998; Walsh et al, 2002; Keayed et al., 2004; Cleary et al., 2005). Furthermore, such Aβ oligomers were found in human liquor (Pitschke et al., 1998; Santos et al., 2007; Klyubin et al., 2008). Besides their neurotoxicity, oligomers have also an influence on the determination of the Aβ concentration in human samples. The oligomerization leads to masking of the C-terminal epitopes of Aβ peptides (Roher et al., 2000) yielding to underestimated Aβ levels detected by C-terminal specific ELISA (Stenh et al., 2005). Thus, the existence of Aβ oligomers in the sample results in lowering of the ELISA signal. This could be a problem for exact determination of the Aβ concentration, however this fact offers also the chance to measure the amount of oligomers and the level of oligomerization in biological samples. The data presented herein surprisingly demonstrates that the content of Aβ oligomers can be determined indirectly by measuring the ELISA signal before and after disaggregation of the oligomers.

The ratio of both values reflect the concentration of soluble Aβ oligomers and the oligomeric level, respectively, in human plasma. Independently from our present invention a similar approach was published very recently (Englund et al., 2009). They determined the Aβ 1-42 oligomer ratio in human CSF samples by measuring the Aβ 1-42 concentration under non-denaturing conditions via ELISA and under denaturing conditions using SDS-PAGE followed by Western Blot analysis. However, this approach of indirect determination of the oligomeric level has some critical issues:

SDS-PAGE is not able to fully disaggregate Aβ 1-42. Our experiences have shown also Aβ trimer and tetramer reflecting bands on the SDS gel.

The comparison of Aβ concentrations determined via ELISA and via Western Blot is defective.

Another more common approach is the direct measurement of Aβ oligomers. Such a method, especially with oligomeric plasma Aβ as a biomarker, is however extremely difficult to establish as the Aβ peptides are very hydrophobic. Currently described assay systems use Aβ oligomer specific antibodies in a ELISA system (Englund et al., 2007; Schupf et al, 2008). However, the usage of ELISAs based on such oligomer specific antibodies have the same problems as traditional Aβ ELISA systems. The methods only achieve very unsatisfactory analytical sensitivity and encounter great problems with the very complex interactions between analytes and matrix, i.e. plasma. Usually, ELISA or ELISA-type systems (Multiplex) are used for quantification of Aβ, and recently also Aβ oligomers, in plasma. The specification of such detections systems is usually only unsatisfactorily analyzed or are completely disregarded. For example a critical item like the recovery rate is not analyzed or is not mentioned in the publications. The recovery rate is however decisive for giving a complete picture of those Aβ peptides or oligomers which occur in plasma. Differences between the studies can also result from the differences in these rates. A further important characteristic of an ELISA or multiplex system is its linearity. Thus, the concentrations determined for the analytes in plasma should only depend on the dilution used in the measurement to a very low degree or not at all. However, this is neither possible for ELISA nor for the multiplex systems for quantification of Aβ in plasma. Thus, the difference between the calculated plasma Aβ (1-42) concentration for a dilution of 1-20 was three times as high as for the 1-2 dilution of the same sample (Hansson et al., 2008). This example alone shows that the use of different dilutions of plasma samples in the several studies makes it impossible to compare the same.

Thus, it is an objective of the present invention to provide a novel method which allows determination of oligomeric Aβ, in particular in plasma, with a high reliability. The present invention uses also the indirect measurement of Aβ oligomers, however, in contrast to the prior art, both values (under denaturing and non-denaturing conditions) were determined with Aβ specific ELISA to ensure the comparability. Because of an initial immunoprecipitation step, which isolates Aβ peptides in monomeric as well as oligomeric form followed by our novel disaggregation method, the subsequent ELISA is not constricted by recovery and/or linearity issues.

Moreover, the present invention aims at providing diagnostic markers which can be determined with reliable methods and can be used for reliable and clear prediction of Alzheimer's disease.

### SUMMARY OF THE INVENTION

According to a first aspect of the invention there is provided a method of diagnosing or monitoring a neurodegenerative disorder, such as Alzheimer's disease and Mild Cognitive Impairment, which comprises determining the oligomeric state of a target amyloid β peptide (Abeta or Aβ) in a biological sample from a test subject, characterized in that said method comprises the following steps:
(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ, which is lower than 1.5 is indicative of a positive diagnosis for a neurodegenerative disorder.

According to a second aspect of the invention there is provided a method of determining the oligomeric state of a target amyloid β peptide (Abeta or Aβ) in a biological sample which comprises the following steps:

(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ, which is in excess of 1, is indicative of the presence of oligomeric Aβ.

Other objects and features will be in part apparent and in part pointed out hereinafter.

### DEFINITIONS

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

“Oligomeric” as used herein refers to a limited number of aggregated Aβ peptide monomer units. Examples of such oligomers include dimers, trimers and tetramers. The term “disaggregation” refers to the process of converting oligomeric forms of Aβ peptide to monomeric forms of Aβ peptide.

“Capture antibody” in the sense of the present application is intended to encompass those antibodies which bind to a target Aβ peptide.

Suitably the capture antibodies bind to the target Aβ peptide with a high affinity. In the context of the present invention, high affinity means an affinity with a K_{D} value of 10⁻⁷M or better, such as a K_{D} value of 10⁻⁸M or better or even more particularly, a K_{D} value of 10⁻⁹M to 10⁻¹²M.

The term “antibody” is used in the broadest sense and specifically covers intact monoclonal antibodies, polyclonal antibodies, multispecific antibodies (e.g. bispecific antibodies) formed from at least two intact antibodies, and antibody fragments as long as they exhibit the desired biological activity. The antibody may be an IgM, IgG (e.g. IgG1, IgG2, IgG3 or IgG4), IgD, IgA or IgE, for example. Suitably however, the antibody is not an IgM antibody. The “desired biological activity” is binding to a target Aβ peptide.

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single determinant on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Köhler et al., Nature, 256:495 (1975), or may be made by generally well known recombinant DNA methods. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain a minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences.

These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986), Reichmann et al, Nature. 332:323-329 (1988): and Presta, Curr. Op. Struct. Biel., 2:593-596 (1992). The humanized antibody includes a Primatized™ antibody wherein the antigen-binding region of the antibody is derived from an antibody produced by immunizing macaque monkeys with the antigen of interest or a “camelized” antibody.

“Single-chain Fv” or “sFv” antibody fragments comprise the V_{H} and V_{L} domains of an antibody, wherein these domains are present in a single polypeptide chain.

Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{D}) in the same polypeptide chain (V_{H}-V_{D}). By using a linker that is too short to allow pairing between the two domains on the same chain, the domains are forced to pair with the complementary domains of another chain and create two antigen-binding sites. Diabodies are described more fully in Hollinger et al., Proc. Natl. Acad. Sol. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In suitable embodiments, the antibody will be purified (1) to greater than 95% by weight of antibody as determined by the Lowry method, and most particularly more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, suitably, silver stain. Isolated antibody includes the antibody in situ within recombinant cells since at least one component of the antibody's natural environment will not be present. Ordinarily, however, isolated antibody will be prepared by at least one purification step.

As used herein, the expressions “cell”, “cell line,” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond.

“Amyloid β, Aβ or β-amyloid” is an in the art recognized term and refers to amyloid β proteins and peptides, amyloid β precursor protein (APP), as well as modifications, fragments and any functional equivalents thereof. In particular, by amyloid β as used herein is meant any fragment produced by proteolytic cleavage of APP but especially those fragments which are involved in or associated with the amyloid pathologies including, but not limited to, Aβ (1-38) of SEQ ID NO. 3, Aβ (1-40) of SEQ ID NO. 2, and Aβ (1-42) of SEQ ID NO. 1.

In the context of the present invention, “fragments of amyloid β” are all amyloid β peptides, which comprise a core amyloid β fragment Aβ (3-38) of SEQ ID NO. 13, More suitably for the purpose of the present invention are all amyloid β peptides, which comprise the core amyloid β fragment Aβ (11-38) of SEQ ID NO. 19. Such Aβ fragments, which comprise the amino acid sequence of Aβ (11-38) of SEQ ID NO. 19, are in particular Aβ (x-y) fragments, which have been shown to accumulate in a subject as a consequence of a neurodegenerative disorder, such as Alzheimer's disease and Mild Cognitive Impairment, wherein
x is defined as an integer selected from 1, 2, 3, 4, 5, 6, 7, 8, 9, 10 and 11;
   Preferably, x is an integer selected from 1, 2, 3 and 11.
   More preferably, x is 1.
   Even more preferably, x is 11.
y is defined as an integer selected from 38, 39, 40, 41, 42 and 43.
   Preferably, y is 38, 40 or 42, such as 40 or 42.
   More preferably, y is 40.
   Even more preferably, y is 38.

Suitable examples for Aβ (x-y) fragments are
Aβ (1-38) (SEQ ID NO. 3),
Aβ (1-39) (SEQ ID NO. 4),
Aβ (1-40) (SEQ ID NO. 2),
Aβ (1-41) (SEQ ID NO. 5)
Aβ (1-42) (SEQ ID NO. 1)
Aβ (1-43) (SEQ ID NO. 6)
Aβ (2-38) (SEQ ID NO. 7),
Aβ (2-39) (SEQ ID NO. 8),
Aβ (2-40) (SEQ ID NO. 9),
Aβ (2-41) (SEQ ID NO. 10),
Aβ (2-42) (SEQ ID NO. 11),
Aβ (2-43) (SEQ ID NO. 12),
Aβ (3-38) (SEQ ID NO. 13),
Aβ (3-39) (SEQ ID NO. 14),
Aβ (3-40) (SEQ ID NO. 15),
Aβ (3-41) (SEQ ID NO. 16),
Aβ (3-42) (SEQ ID NO. 17),
Aβ (3-43) (SEQ ID NO. 18),
Aβ (11-38) (SEQ ID NO. 19),
Aβ (11-39) (SEQ ID NO. 20),
Aβ (11-40) (SEQ ID NO. 21),
Aβ (11-41) (SEQ ID NO. 22),
Aβ (11-42) (SEQ ID NO. 23), and
Aβ (11-43) (SEQ ID NO. 24).

“Functional equivalents” encompass all those mutants or variants of Aβ (x-y) which might naturally occur in the patient group which has been selected to undergo the method for detection or method for diagnosis as described according to the present invention. More particularly, “functional equivalent” in the present context means that the functional equivalent of Aβ (x-y) are mutants or variants thereof and have been shown to accumulate in Alzheimer's disease. The functional equivalents have no more than 30, such as 20, e.g. 10, particularly 5 and most particularly 2, or only 1 mutation(s) compared to the respective Aβ (x-y) peptide. Functional equivalents also encompass mutated variants, which comprise by way of example all Aβ peptides starting with amino acids Asp-Ala-Glu and ending with Gly-Val-Val and Val-Ile Ala, respectively.

Particularly useful equivalents in the present context are those of Aβ (1-40) (SEQ ID NO. 2) and Aβ (1-42) (SEQ ID NO. 1), which are those described by Irie et al., 2005, namely the Tottori, Flemish, Dutch, Italian, Arctic and Iowa mutations of Aβ. Functional equivalents also encompass Aβ peptides derived from amyloid precursor protein bearing mutations next to the β- or γ-secretase cleavage site such as the Swedish, Austrian, French, German, Florida, London, Indiana and Australian variations (Irie et al., 2005).

“Modified Amyloid β, Aβ or β-amyloid” encompasses all modifications at various amino acid positions in the amyloid β proteins and peptides, amyloid β precursor protein (APP), fragments and functional equivalents thereof. Useful in the present context are modifications at the N- and/or C-terminal amino acids of said amyloid β proteins and peptides, amyloid β precursor protein (APP), fragments and functional equivalents. Particularly useful are modifications at glutamine and glutamate residues, such as the cyclization of N-terminal glutamine or glutamate residues to pyroglutamate. Suitable examples according to the present invention are the amyloid β peptides of SEQ ID Nos. 13 to 24, which start with a glutamate residue at the N-terminus, wherein said the N-terminal glutamate residue is modified to pyroglutamate. Even useful are modifications at aspartate residues, such as the conversion of asparte to isoaspartate. Suitable examples according to the present invention are the amyloid β peptides of SEQ ID Nos. 1 to 6, wherein the aspartate residues at amino acid positions 1 and/or 7 are converted to isoasparate. Further suitable examples are the amyloid β peptides of SEQ ID Nos. 7 to 12, wherein the aspartate residue at amino acid position 6 is converted to isoasparate. Moreover, suitable examples are the amyloid β peptides of SEQ ID Nos. 13 to 18, wherein the aspartate residue at amino acid position 5 is converted to isoasparate.

“Sandwich ELISAs” usually involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. The second antibody may itself be labeled with a detectable moiety (direct sandwich assays) or may be measured using an anti-immunoglobulin antibody that is labeled with a detectable moiety (indirect sandwich assay). For example, one suitable type of sandwich assay is an ELISA assay, in which case the detectable moiety is an enzyme.

### DESCRIPTION OF THE DRAWINGS

Those of skill in the art will understand that the drawings, described below, are for illustrative purposes only. The drawings are not intended to limit the scope of the present teachings in any way.

FIG. 1 shows a bivalent immunoprecipitation system improves capture efficiency.

FIG. 1A shows recovery of Aβ 1-40 from Cyp18 solution and human plasma by usage of different antibody combinations. FIG. 1B shows a schematic of bivalent capture system (shaded: 4G8 antibody, grey: x-40 antibody, black: anti-mouse antibody conjugated to magnetic bead).

FIG. 2 shows determination of the oligomeric state of Aβ peptides derived from human plasma. The ratio of concentration determined after disaggregation to the concentration determined without disaggregation reflects the oligomeric state of Aβ (1-40/42).

FIG. 3 shows results of a DemTect test. Mean values (Mean±SD) of the results of classification differences in AD patients and healthy subjects (Group I: 18-30 years; Group II: 31-45 years; Group III: 46-65 years) by DemTect Scale.

FIG. 4 shows results of a Mini-Mental-State test. Mean values (Mean±SD) of the results of classification differences in AD patients and healthy subjects (Group I: 18-30 years; Group II: 31-45 years; Group III: 46-65 years) by Mini-Mental-State Test.

FIG. 5 shows results of a Clock-Drawing test. Mean values (Mean±SD) of the results of classification differences in AD patients and healthy subjects (Group I: 18-30 years; Group II: 31-45 years; Group III: 46-65 years) by Clock-Drawing Test.

FIG. 6 shows mean values of oligomeric state, T-test of AD group vs. Control group. ** T-test, p<0.01; *** T-test, p<0.001.

FIG. 7 shows oligomeric state of Aβ (1-40)+Aβ (1-42) as reflection of overall oligomeric amount in plasma.

### DETAILED DESCRIPTION OF THE INVENTION

According to a first aspect of the invention there is provided a method of diagnosing or monitoring a neurodegenerative disorder, such as Alzheimer's disease and Mild Cognitive Impairment, which comprises determining the oligomeric state of a target amyloid β peptide (Abeta or Aβ) in a biological sample from a test subject, characterized in that said method comprises the following steps:
(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ, which is lower than 1.5 is indicative of a positive diagnosis for a neurodegenerative disorder.

The data presented herein surprisingly demonstrate that the oligomeric state of Aβ was significantly decreased in Alzheimer's disease patients when compared with control patients. Therefore, the oligomeric state of Aβ appears to be a reliable and clear prediction of Alzheimer's disease. A ratio (c_{d}/cₐ) of 1.0 indicates that there are no oligomers in the sample. Higher ratios of c_{d}/cₐ (i.e. ratios of >1.0) reflect a greater amount of oligomers or more compactness of oligomers (less accessibility of epitopes) in the sample. Ratios of c_{d}/cₐ, which are lower than 1.5 (i.e. a ratio between 1.0 and 1.5), such as lower than 1.4, lower than 1.3, lower than 1.2, lower than 1.1 or lower than 1.05 have been found to be indicative of a positive diagnosis for a neurodegenerative disorder, such as Alzheimer's disease.

In another embodiment of the invention there is provided a method of diagnosing or monitoring a neurodegenerative disorder, such as Alzheimer's disease and Mild Cognitive Impairment, which comprises determining the oligomeric state of a target amyloid β peptide (Abeta or Aβ) in a biological sample from a test subject, characterized in that said method comprises the following steps:
(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) adding up the values of c_{d} and cₐ, wherein the value of the sum of c_{d} and cₐ, which is lower than 3.0, is indicative of a positive diagnosis for a neurodegenerative disorder.

A sum of c_{d} and cₐ, which is lower than 2.9, lower than 2.8, lower than 2.7, lower than 2.6, lower than 2.5, lower than 2.4 or lower than 2.3 have been found to be indicative of a positive diagnosis for a neurodegenerative disorder, such as Alzheimer's disease.

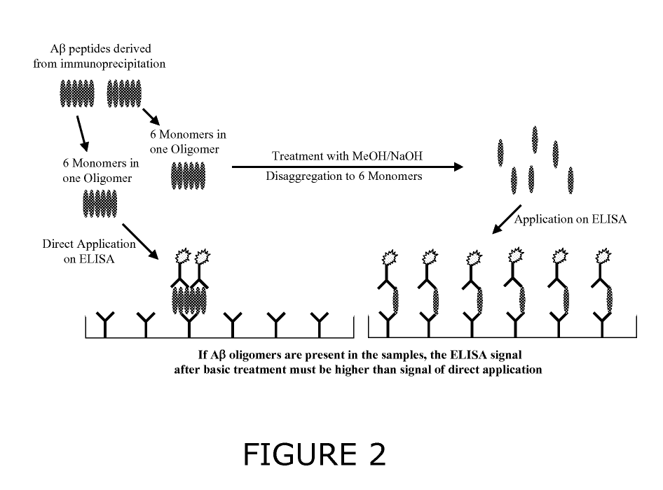

In nearly all studies, the concentration of target Aβ peptides in steps (a) and (c) was determined by sandwich ELISA systems consisting of a capture and a detection antibody. Compared with the size of an antibody (150 kDa), Aβ peptides (4.5 kDa) as monomers are very small. Because of the aggregation propensity of the peptides they tend to form oligomers, protofibrils to the point of fibrils. Within such an aggregate the Aβ monomers are tightly packed with the consequence that not all monomers can be bound by the detection antibody due to sterical hindrance or epitope inaccessibility. The detected Aβ concentration for oligomers is lower than for monomers. This would lead to underestimated Aβ levels in plasma and CSF, respectively. The discrepancy between measured and actual concentration is dependent upon the amount of oligomers and their compactness. Inversely, the amount of Aβ aggregates, or more precisely the burden epitopes, can be determined by comparison of the concentration detected in the presence of oligomers and the concentration after disaggregation of oligomers completely to monomers. The principle of the method is shown in FIG. 2.

In one embodiment, the disaggregation step (b) comprises the use of an alkali.

In a further embodiment, the alkali used for disaggregation in step (b) is sodium hydroxide, such as 500 mM sodium hydroxide. The advantage of using an alkali and particularly a strong alkali such as sodium hydroxide is that more efficient disaggregation is achieved. For example, a higher proportion of monomers are obtained, with no observable quantities of dimers, trimers or tetramers.

In one embodiment, the disaggregation step (b) additionally comprises the use of a suitable solvent, such as methanol, e.g. 50% (v/v) methanol.

In one embodiment, the disaggregation step (b) comprises an incubation step. In a further embodiment, the incubation step comprises incubation at room temperature for at least 2 minutes. In a yet further embodiment, the incubation step comprises incubation at room temperature for at least 10 minutes.

Aβ peptides are liberated from the amyloid precursor protein (APP) after a sequential cleavage by the enzymes β- and γ-secretase. The γ-secretase cleavage results in the generation of primarily Aβ (1-40) and Aβ (1-42) peptides but also ending prominently at position 38 or 43, which differ in their C-termini and exhibit different potencies of aggregation, fibril formation and neurotoxicity. Also, β-secretase release can generate different N-termini and also subsequent modifications by peptidases and other enzymes resulting in prominent species such as Aβ peptides starting at the positions e.g. 2, 3, 4 and also 11, while the species staring at positions glutamate 3 and 11 can be transformed into pyroglutamate, rendering these peptides especially hydrophobic and prone to fast aggregation (Schilling et al, 2004; Piccini et al., 2005; Schilling et al, 2006; Schlenzig et al, 2009). Such C- and N-terminal variants of Aβ can serve as functional equivalents of Aβ (1-40) and Aβ (1-42) peptides.

The present invention thus provides a method for the determination of the oligomeric states of the Aβ (x-y) peptides, wherein x and y are as hereinbefore defined.

Thus, according to one embodiment of the above-described method, the oligomeric state of a target AR peptide to be determined is selected from the group consisting of:
Aβ (1-38) (SEQ ID NO. 3),
Aβ (1-39) (SEQ ID NO. 4),
Aβ (1-40) (SEQ ID NO. 2),
Aβ (1-41) (SEQ ID NO. 5)
Aβ (1-42) (SEQ ID NO. 1)
Aβ (1-43) (SEQ ID NO. 6)
Aβ (2-38) (SEQ ID NO. 7),
Aβ (2-39) (SEQ ID NO. 8),
Aβ (2-40) (SEQ ID NO. 9),
Aβ (2-41) (SEQ ID NO. 10),
Aβ (2-42) (SEQ ID NO. 11),
Aβ (2-43) (SEQ ID NO. 12),
Aβ (3-38) (SEQ ID NO. 13),
Aβ (3-39) (SEQ ID NO. 14),
Aβ (3-40) (SEQ ID NO. 15),
Aβ (3-41) (SEQ ID NO. 16),
Aβ (3-42) (SEQ ID NO. 17),
Aβ (3-43) (SEQ ID NO. 18),
Aβ (11-38) (SEQ ID NO. 19),
Aβ (11-39) (SEQ ID NO. 20),
Aβ (11-40) (SEQ ID NO. 21),
Aβ (11-41) (SEQ ID NO. 22),
Aβ (11-42) (SEQ ID NO. 23), and
Aβ (11-43) (SEQ ID NO. 24).

In a particular embodiment, the oligomeric state of a target Aβ peptide to be detected is Aβ (1-40) (SEQ ID No: 2).

In a particular embodiment, the oligomeric state of a target Aβ peptide to be detected is Aβ (1-42) (SEQ ID No: 1).

In a particular embodiment, the oligomeric state of a target Aβ peptide to be detected is Aβ (1-40) (SEQ ID No: 2) and Aβ (1-42) (SEQ ID No: 1). The data presented herein demonstrates the suitability of summation of the Aβ (1-40) and Aβ (1-42) peptides wherein it has been shown that summation of both oligomeric states improves the significance of the diagnosis.

In a particular embodiment, the oligomeric state of a target Aβ peptide to be detected is at least one Aβ peptide selected from the SEQ ID NOs: 13 to 24, which start with a glutamate residue at the N-terminus.

In a particular embodiment, the oligomeric state of a target Aβ peptide to be detected is Aβ (3-38) (SEQ ID No: 13).

In a particular embodiment, the oligomeric state of a target Aβ peptide to be detected is Aβ (11-38) (SEQ ID No: 19).

In a further particular embodiment, the oligomeric state of a target Aβ peptide to be detected is at least one Aβ peptide selected from SEQ ID NOs: 13 to 24, wherein the glutamate residue at the N-terminus of these peptides is cyclized to pyroglutamate.

In a further particular embodiment, the oligomeric state of a target Aβ peptide to be detected is at least one Aβ peptide selected from SEQ ID Nos. 1 to 6, wherein the aspartate residues at amino acid positions 1 and/or 7 are converted to isoasparate.

Even particularly, the oligomeric state of a target Aβ peptide to be detected is at least one Aβ peptide selected from SEQ ID Nos. 7 to 12, wherein the aspartate residue at amino acid position 6 is converted to isoasparate.

Even particularly, the oligomeric state of a target Aβ peptide to be detected is at least one Aβ peptide selected from SEQ ID Nos. 13 to 18, wherein the aspartate residue at amino acid position 5 is converted to isoasparate.

It will be appreciated that the method of determining the oligomeric state of Aβ constitutes a further aspect of the invention related to a novel and inventive assay which is not necessarily limited to the diagnosis of a neurodegenerative disorder such as Alzheimer's disease. Thus, according to a second aspect of the invention there is provided a method of determining the oligomeric state of a target amyloid β peptide (Abeta or Aβ) in a biological sample which comprises the following steps:
(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ, which is in excess of 1, is indicative of the presence of oligomeric Aβ.

In one embodiment, the disaggregation step (b) comprises the use of an alkali as hereinbefore defined.

In one embodiment, the method of determining the first and second concentration of a target Aβ peptide in steps (a) and (c) comprise:
i) contacting a biological sample with at least two different capture antibodies,
ii) detection of the resulting immune complex,
iii) destruction of the immune complex, and,
iv) quantifying the captured Aβ peptides.

In one embodiment, the quantifying step (iv) comprises analysis in an Aβ specific ELISA. In a further embodiment, the Aβ specific ELISA is a sandwich-ELISA. In one embodiment, steps (a) and (c) both comprise analysis with an Aβ specific ELISA. This embodiment provides the advantage of allowing comparability between the first and second concentrations obtained in steps (a) and (c).

In one embodiment, the biological sample is selected from the group consisting of blood, serum, urine, cerebrospinal fluid (CSF), plasma, lymph, saliva, sweat, pleural fluid, synovial fluid, tear fluid, bile and pancreas secretion. In a further embodiment, the biological sample is plasma.

The biological sample can be obtained from a patient in a manner well-known to a person skilled in the art. In particular, a blood sample can be obtained from a subject and the blood sample can be separated into serum and plasma by conventional methods. The subject, from which the biological sample is obtained is suspected of being afflicted with Alzheimer's disease, at risk of developing Alzheimer's disease and/or being at risk of or having any other kind of dementia.

In particular, it is a subject suspected of having Mild Cognitive Impairment (MCI) and/or being in the early stages of Alzheimer's disease.

The present method has several advantages over the methods known in the art, i.e. the method of the present invention can be used to detect Alzheimer's disease at an early stage and to differentiate between Alzheimer's disease and other types of dementia in early stages of disease development and progression. One possible early stage is Mild Cognitive Impairment (MCI). It is impossible with the methods currently known in the art to make a clear and reliable diagnosis of early stages of Alzheimer's disease and, in particular, it is impossible to differentiate between the onset of Alzheimer's disease and other forms of dementia in said early stages. This especially applies for patients afflicted with MCI.

In contrast, the methods provided by the present invention are suitable for a differential diagnosis of Alzheimer's disease. In particular, the present invention provides a method, wherein the oligomeric state of target Aβ peptides can be detected in biological samples obtained from any of the above described subjects in a highly reproducible manner. The high reproducibility of the methods of the present invention is achieved by using at least two different capture antibodies in an initial immune-precipitation step (step (a)) which is identical to the process subsequently used in step (c). In one embodiment, these at least two different capture antibodies are directed to different epitopes of the Aβ target peptide. In one embodiment, the biological sample is plasma.

The above-mentioned “Aβ target peptide” encompasses Aβ (x-y) as hereinbefore defined.

A specific problem, which had to be overcome by the present invention, is that the biomarker to be used is altered in early stages of Alzheimer's disease, e.g. during mild cognitive impairment. The inventors of present invention have shown that it is possible to determine the oligomeric state of target Aβ peptides, in a reliable manner, and, it also became clear for the first time that in fact the oligomeric state of Aβ (x-y) is particularly suitable for the diagnosis of early onset Alzheimer's disease.

The method of determining the first and second concentration of a target Aβ peptide in steps (a) and (c) specifically comprises the following steps:
i) Contacting a biological sample with at least two different capture antibodies in an immunoprecipitation step.

After contacting the biological sample with the aforementioned at least two different capture antibodies, an immune complex will form between the at least two different capture antibodies and the target Aβ peptides. This step does not act for specific isolation of full length Aβ (x-y) wherein x would be 1, rather than capturing and separating all Aβ species, especially ending at position 38, 40 and/or 42.
ii) This complex is then detected by secondary antibodies. Suitably, the secondary antibodies are immobilized on magnetic beads. Together with the magnetic beads the immune complex can be easily separated from the body fluid (plasma/serum CSF etc.) using the magnetic separator.
iii) The immune complex is eluted from the beads. Suitably, the elution step is performed by incubating the beads carrying the immune complex in a solution comprising 50% Methanol/0.5% formic acid for 1 h at room temperature. Thereby, all intermolecular interactions are destructed and all Aβ peptide molecules, which were isolated from the biological sample, are released from the beads in the solution.
iv) The released, isolated Aβ peptide will be quantified in a subsequent step, for example by a sandwich ELISA that specifically detects full length Aβ (x-y), wherein full length Aβ (x-y) in this step most suitably means Aβ (1-40) and Aβ (1-42).

Possible antibodies for immunoprecipitation, which would be suitable in the present context, are the following, although the present invention is not delimited to those specific working examples:
3D6, Epitope: 1-5 (Elan Pharmaceuticals, Innogenetics)
pAb-EL16, Epitope: 1-7
2H4, Epitope: 1-8 (Covance)
1E11, Epitope: 1-8 (Covance)
20.1, Epitope: 1-10 (Covance, Santa Cruz Biotechnology)
Rabbit Anti-Aβ Polyclonal Antibody, Epitope: 1-14 (Abcam)
AB10, Epitope: 1-16 (Chemicon/Upstate—part of Millipore)
82E1, Epitope: 1-16 (IBL)
pAb 1-42, Epitope: 1-11
NAB228, Epitope: 1-11 (Covance, Sigma-Aldrich, Cell Signaling, Santa Cruz Biotechnology, Zymed/Invitrogen)
DE2, Epitope: 1-16 (Chemicon/Upstate—part of Millipore)
DE2B4, Epitope: 1-17 (Novus Biologicals, Abcam, Accurate, AbD Serotec)
6E10, Epitope: 1-17 (Signet Covance, Sigma-Aldrich)
10D5, Epitope: 3-7 (Elan Pharmaceuticals)
WO-2, Epitope: 4-10 (The Genetics Company)
1A3, Epitope 5-9 (Abbiotec)
pAb-EL21, Epitope 5-11
310-03, Epitope 5-16 (Abcam, Santa Cruz Biotechnology)
Chicken Anti-Human Aβ Polyclonal Antibody, Epitope 12-28 (Abcam)
Chicken Anti-Human Aβ Polyclonal Antibody, Epitope 25-35 (Abcam)
Rabbit Anti-Human Aβ Polyclonal Antibody, Epitope: N-terminal (ABR)
Rabbit Anti-Human Aβ Polyclonal Antibody (Anaspec)
12C3, Epitope 10-16 (Abbiotec, Santa Cruz Biotechnology)
16C9, Epitope 10-16 (Abbiotec, Santa Cruz Biotechnology)
19B8, Epitope 9-10 (Abbiotec, Santa Cruz Biotechnology)
pAb-EL26, Epitope: 11-26
BAM90.1, Epitope: 13-28 (Sigma-Aldrich)
Rabbit Anti-beta-Amyloid (pan) Polyclonal Antibody, Epitope: 15-30 (MBL)
22D12, Epitope: 18-21 (Santa Cruz Biotechnology)
266, Epitope: 16-24 (Elan Pharmaceuticals)
pAb-EL17; Epitope: 15-24
4G8, Epitope: 17-24 (Covance)
Rabbit Anti-Aβ Polyclonal Antibody, Epitope: 22-35 (Abcam)
G2-10; Epitope: 31-40 (The Genetics Company)
Rabbit Anti-Aβ, aa 32-40 Polyclonal Antibody (GenScript Corporation)
EP1876Y, Epitope: x-40 (Novus Biologicals)
G2-11, Epitope: 33-42 (The Genetics Company)
16C11, Epitope: 33-42 (Santa Cruz Biotechnology)
21F12, Epitope: 34-42 (Elan Pharmaceuticals, Innogenetics)
1A10, Epitope: 35-40 (IBL)
D-17 Goat anti-Aβ antibody, Epitope: C-terminal (Santa Cruz Biotechnology).

Particular antibodies for the immunoprecipitation are: 3D6 (Elan), BAN50 (Takeda), 82E1 (IBL), 6E10 (Covance), WO-2 (The Genetics Company), 266 (Elan), BAM90.1 (Sigma), 4G8 (Covance), G2-10 (The Genetics Company), 1A10 (IBL), BA27 (Takeda), 11A5-B10 (Millipore), 12F4 (Millipore), 21F12 (Elan).

Examples for AβN3pE specific antibodies are:
the Pyro-Glu Abeta antibodies Aβ 5-5-6 (Deposit No. DSM ACC 2923), Aβ 6-1-6 (Deposit No. DSM ACC 2924)
Aβ 17-4-3 (Deposit No. DSM ACC 2925) and Aβ 24-2-3 (Deposit No. DSM ACC 2926), which are described in PCT/EP2009/058803 (monoclonal, mouse), Probiodrug AG
Pyro-Glu Abeta antibody clone 2-48 (monoclonal, mouse); Synaptic Systems
Pyro-Glu Abeta antibody (polyclonal, rabbit); Synaptic Systems
Pyro-Glu Abeta antibody clone 8E1 (monoclonal, mouse); Anawa
Pyro-Glu Abeta antibody clone 8E1 (monoclonal, mouse); Biotrend
Anti-Human Amyloidβ (N3pE) Rabbit IgG (polyclonal, rabbit); IBL
Anti-Human Aβ N3pE (8E1) Mouse IgG Fab (monoclonal, mouse); IBL

Examples for Aβ isoAsp 1 specific antibodies are:
anti-human Aβ isoAsp 1 antibody (polyclonal, rabbit); disclosed in Saido T C, et al., Neurosci Lett. (1996) 13; 215(3):173-6.

Particular antibody pairs for the immunoprecipitation are:

4G8 and 11A5-B10, 3D6 and 4G8, 6E10 and 4G8, 82E1 and 4G8, 4G8 and 12F4, 4G8 and 21F12, 3D6 and 21F12, 6E10 and 21F12, BAN50 and 4G8, 3D6 and 11A5-B10, 3D6 and 1A10, 3D6 and BA27, 6E10 and 11A5-B10, 6E10 and 1A10, 6E10 and BA27, 4G8 and 11A5-B10, 4G8 and 1A10, 4G8 and BA27, 4G8 and 12F4, 4G8 and 21F12.

Examples for AβN3pE specific antibodies are:
the Pyro-Glu Abeta antibodies Aβ 5-5-6 (Deposit No. DSM ACC 2923), Aβ 6-1-6 (Deposit No. DSM ACC 2924)
Aβ 17-4-3 (Deposit No. DSM ACC 2925) and Aβ 24-2-3 (Deposit No. DSM ACC 2926), which are described in PCT/EP2009/058803 (monoclonal, mouse), Probiodrug AG
Pyro-Glu Abeta antibody clone 2-48 (monoclonal, mouse); Synaptic. Systems
Pyro-Glu Abeta antibody (polyclonal, rabbit); Synaptic Systems
Pyro-Glu Abeta antibody clone 8E1 (monoclonal, mouse); Anawa
Pyro-Glu Abeta antibody clone 8E1 (monoclonal, mouse); Biotrend
Anti-Human Amyloidβ (N3pE) Rabbit IgG (polyclonal, rabbit); IBL
Anti-Human Aβ N3pE (8E1) Mouse IgG Fab (monoclonal, mouse); IBL

Examples for Aβ isoAsp 1 specific antibodies
anti-human Aβ isoAsp 1 antibody (polyclonal, rabbit); Saido et al., 1996).

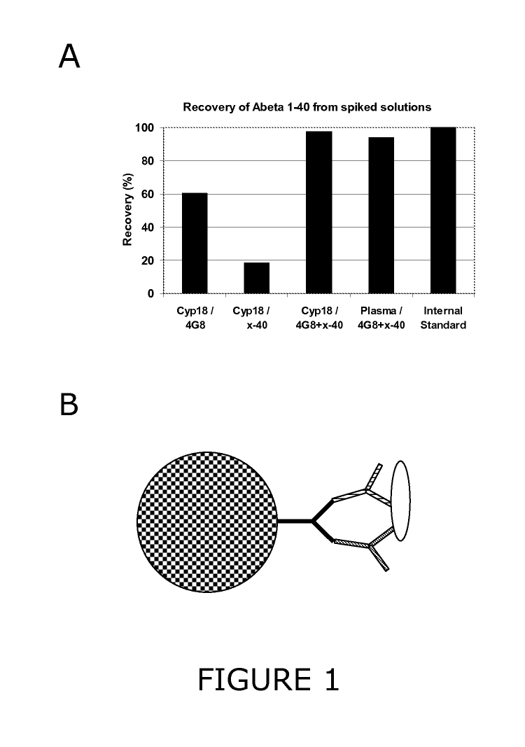

Apart from the above designated antibodies, all other amyloid beta specific antibodies (monoclonal and polyclonal) which are suitable for immunoprecipitation can be used in the concentration determining method (further suitable antibodies can, for example, be taken from alzforum.org). Decisive for good capture efficiency is the use of two, three or more different antibodies with different epitopes. The use of more than one antibody type for immunoprecipitation of Aβ peptides can offer cooperative and surprisingly synergistic binding effects (avidity), which finally allows to achieve a tremendously higher capture efficiency (see FIG. 1).

The secondary antibodies in step ii) are specific against the host antibody type of the capture antibodies. Suitable secondary antibodies are anti-mouse antibodies and anti-rabbit antibodies.

After incubation of the complex with the magnetic beads in step iii), the beads can be washed with washing buffer (see Examples disclosed herein). Washing buffers, which contain detergents or other additives preventing unspecific binding, can be used for this step. Non-limiting examples of washing buffers include:
D-PBS containing 10 mg/ml Cyclophilin 18 (Cyp 18) and 0.05% Tween-20,
PBS+0.05% Tween-20,
TBS+0.05% Tween-20,
PBS+1% (w/v) BSA+0.05% Tween-20,
TBS+1% (w/v) BSA+0.05% Tween-20, and
Pierce ELISA Blocker (with Tween-20).

After elution of the immune complex from the beads in step iv), the solution is diluted in dilution buffer. Any dilution buffers, which can prevent unspecific interaction with surfaces and the immobilized first ELISA antibody can be used for this step. Non-limiting examples for dilution buffers are:
EIA buffer (dilution buffer of the IBL 1-40 (N) ELISA Kit),
PBS+1% (w/v) BSA+0.05% Tween-20,
TBS+1% (w/v) BSA+0.05% Tween-20, and
Pierce ELISA Blocker (with Tween-20).

ELISA-Kits that are able to quantify full length Aβ (1-40) are commercially available. Suitable ELISA-Kits for the quantification of Aβ (1-40) in the methods of the present invention are for example: Amyloid-β (1-40) (N) ELISA (IBL, JP27714); Aβ [1-40] Human ELISA Kit (Invitrogen); Human Amyloid beta (Amyloid-β), aa 1-40 ELISA Kit (Wako Chemicals USA, Inc.); Amyloid Beta 1-40 ELISA Kit (The Genetics Company).

ELISA-Kits that are able to quantify full length Aβ (1-42) are also commercially available. Suitable ELISA-Kits for the quantification of Aβ (1-42) in the methods of the present invention are for example: Amyloid-β (1-42) (N) ELISA (IBL, JP27712); Aβ [1-42] Human ELISA Kit (Invitrogen), Human Amyloid beta (Amyloid-β), aa 1-42 ELISA Kit (Wako Chemicals USA, Inc.), Amyloid Beta 1-40 ELISA Kit (The Genetics Company), INNOTEST® β-AMYLOID (1-42) (Innogenetics).

The concentration determining method is not limited to the exemplary aforementioned commercially available ELISA-Kits for Aβ (1-40) or Aβ (1-42). Numerous further sandwich ELISAs for full length Aβ (1-40) or Aβ (1-42) may be available in the prior art or may be developed by the skilled artisan. All these full length Aβ 1-40 or Aβ 1-42 sandwich ELISAs shall also be encompassed by the concentration determining method and should typically comprise a suitable pair of capture and detection antibodies, which are specific for the complete N-terminus of Aβ (1-40) and/or Aβ (1-42) and the C-terminus ending at amino acid 40 or 42, respectively.

Such a full length Aβ (1-40) sandwich ELISA may comprise a first immobilized antibody recognizing specifically the C-terminus of Aβ (1-40) and a second labeled detection antibody recognizing specifically the complete N-terminus of Aβ (1-40).

A full length Aβ (1-42) sandwich ELISA may comprise a first immobilized antibody recognizing specifically the C-terminus of Aβ (1-42) and a second labeled detection antibody recognizing specifically the complete N-terminus of Aβ (1-42).

A full length Aβ (1-40) sandwich ELISA may also comprise a first immobilized antibody recognizing specifically the complete N-terminus of Aβ (1-40) and a second labeled detection antibody recognizing specifically the C-terminus of Aβ (1-40).

A full length Aβ (1-42) sandwich ELISA may also comprise a first immobilized antibody recognizing specifically the complete N-terminus of Aβ (1-42) and a second labeled detection antibody recognizing specifically the C-terminus of Aβ (1-42).

Suitable Aβ (1-40/42) N-terminal specific antibodies for use in the concentration determining method are for example 3D6 (Elan), WO-2 (The Genetics Company), 82E1 (IBL), BAN-50 (Takeda). Numerous further Aβ (1-40/42) N-terminal specific antibodies may be available in the prior art or may be developed by the skilled artisan. All these Aβ (1-40/42) N-terminal specific antibodies are also envisaged for the concentration determining method.

Suitable Aβ (1-40) C-terminal specific antibodies are for example G2-10 (The Genetics Company); 11A5-B10 (Millipore); 1A10 (IBL); BA27 (Takeda); EP1876Y (Novus Biologicals). Numerous further Aβ (1-40) C-terminal specific antibodies may be available in the prior art or may be developed by the skilled artisan. All these Aβ (1-40) C-terminal specific antibodies are also envisaged for the concentration determining method.

Suitable Aβ (1-42) C-terminal specific antibodies are for example G2-11 (The Genetics Company); 12F4 (Millipore); Anti-Human Aβ (38-42) Rabbit IgG (IBL); 21F12 (Elan); BC05 (Takeda); 16C11 (Santa Cruz Biotechnology). Numerous further Aβ (1-42) C-terminal specific antibodies may be available in the prior art or may be developed by the skilled artisan. All these Aβ (1-42) C-terminal specific antibodies are envisaged for the concentration determining method.

According to one embodiment, the detection antibodies are labeled.

For diagnostic applications, the detection antibody will typically be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Gütigen et al., Ed., Wiley-Interscience. New York, N.Y. Pubs., (1991) for example and radioactivity can be measured using scintillation counting.
(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra for example. Fluorescence can be quantified using a fluorimeter.
(c) Various enzyme-substrate labels are available. The enzyme generally catalyses a chemical alteration of the chromogenic substrate which can be measured using various techniques. For example, the enzyme may catalyze a colour change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g, firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase. O-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed Langone & H. Van Vunakis), Academic Press, New York, 73: 147-166 (1981).

Examples of enzyme-substrate combinations include, for example:
(i) Horseradish peroxidase (HRPO) with hydrogen peroxidase as a substrate, wherein the hydrogen peroxidase oxidizes a dye precursor (e.g. orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (6-D-Gal) with a chromogenic substrate (e.g. p-nitrophenyl-β-D-galactosidase) or the fluorogenic substrate 4-methylumbelliferyl-β-D-galactosidase.

Numerous other enzyme-substrate combinations are available to those skilled in the art.

(d) Another possible label for a detection antibody is a short nucleotide sequence. The concentration is then determined by a RT-PCR system (Imperacer™, Chimera Biotech).

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

The antibodies used in the present invention may be employed in any known assay method, such as competitive binding assays, direct and indirect sandwich assays, and immunoprecipitation assays. Zola, Monoclonal Antibodies A Manual of Techniques, pp. 147-158 (CRC Press. Inc., 1987).

Competitive binding assays rely on the ability of a labeled standard to compete with the test sample analyte for binding with a limited amount of antibody. The amount of Aβ peptide in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

For the analysis of the Aβ (1-40) concentration in human all of the following body fluids can be used: blood, cerebrospinal fluid (CSF), urine, lymph, saliva, sweat, pleural fluid, synovial fluid, aqueous fluid, tear fluid, bile and pancreas secretion.

The novel method was established by the present inventors using blood samples (see the examples of the present invention). The present method is however not to be construed to be limited to blood samples. The method can also be employed using CSF, brain extract and urine samples, as well as all other human body fluids, e.g. the above mentioned in the same manner. Particular samples include plasma samples.

For immunohistochemistry analyses, the tissue sample may be fresh or frozen or may be embedded in paraffin and fixed with a preservative such as formalin, for example.

It will be appreciated that although the sandwich ELISA system comprises one particular embodiment for determining Aβ concentration in steps (a) and (c) of the invention, other concentration determining methods may be used.

Suitable alternative methods for determining the concentration of Aβ are:

### 1. Amyloid β 1-40 HTRF® Assay (CisBio Bioassays):

This assay principle is based on TR-FRET, which is a combination of Time-Resolved Fluorescence and Förster Resonance Energy Transfer. Similar to the usual sandwich ELISA the Aβ (1-40) is bound by two antibodies; the antibodies are here, however, not bound on a surface, the interaction occurs in solution. Both antibodies are labeled with a fluorophor. When these two fluorophors are brought together by a biomolecular interaction a portion of energy captured by the donor fluorophor during excitation is transferred via FRET to an acceptor fluorophor, which will be excited as a result. The fluorescence of the acceptor fluorophor is measured. The measuring signal is correlated with the amount of FRET and thus, the amount of Aβ (1-40) in solution.

Similarly, based on a comparable principle, the Alphascreen™ Assay from Lilly can be used.

### 2. Multiplex Assay Systems

Multiplex Assay Systems are available from several manufacturers and are well known and broadly used in the field. A suitable example for use in the methods of the present invention is the INNO-BIA plasma Aβ forms assay (Innogenetics). This assay is a well standardized multiparameter bead-based immunoassay for the simultaneous quantification of human β-amyloid forms Aβ (1-42) and Aβ (1-40) or Aβ(X-42) and Aβ(X-40) in plasma using xMAP® technology (xMAP is a registered trademark of Luminex Corp.).

This assay system is able to quantify up to 100 different analytes in parallel. The basis of this method are small spherical polystyrol particles, called microspheres or beads. In analogy to ELISA and Western Blot these beads serve as a solid phase for the biochemical detection. These beads are colour-coded, so that 100 different bead classes can be distinguished. Every bead class has one specific antibody (e.g. against Aβ (1-40)) immobilized on the microsphere surface. If the Aβ (1-40) concentration increases more peptide molecules will be bound by the beads of this class. The detection of the binding of the analyte is carried out by a second anti-Aβ (1-40) antibody, which is labeled with another fluorescence dye, emitting green light. The sample is handled comparable to FACS analysis. The microspheres are singularized by hydrodynamic focusing and analyzed by laser-based detection system, which can make a quantification on the basis of the green fluorescence and identify the bound analyte by the specific coloration of the bead. Thus, it is possible to determine the concentration of multiple analytes in one sample.

3. Quantification by mass spectrometry
For quantification of Aβ (1-40) also the SELDI-TOF mass spectrometry was used (Simonsen et al., 2007 (2)).
Quantitative analysis of Aβ peptides using immunoprecipitation and MALDI-TOF mass spectrometry. ¹⁵N labeled standard Aβ peptides are used for calibration. (Gelfanova et al., 2007).

### 4. Western Blot Analysis

2D-Gel electrophoresis coupled with Western Blot analysis may be a suitable method to quantify Aβ peptides (Sergeant et al., 2003; Casas et al., 2004).

### Diagnostic Kits

As a matter of convenience, the antibodies used in the method of the present invention can be provided in a kit, i.e., a packaged combination of reagents in predetermined amounts with instructions for performing the diagnostic assay.

Thus, according to a further aspect of the invention there is provided a kit for diagnosing a neurodegenerative disorder, such as Alzheimer's disease which comprises a suitable alkali and instructions to use said kit in accordance with the methods defined herein.

In one embodiment, the kit additionally comprises at least two different capture antibodies as defined herein.

Where the antibody is labeled with an enzyme, the kit will include substrates and cofactors required by the enzyme (e.g. a substrate precursor which provides the detectable chromophore or fluorophore). In addition, other additives may be included such as stabilizers, buffers (e.g. a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

The diagnostic kit of the invention is especially useful for the detection and diagnosis of neurodegenerative disorders, such as amyloid-associated diseases and conditions, e.g. Alzheimer's disease.

### Uses

The method of the present invention makes it possible for the first time to detect and quantify oligomeric target Aβ peptides, in particular Aβ (1-40), Aβ (1-42), Aβ (3-38) and/or Aβ (11-38), or a functional equivalent thereof, in a reliable manner. In particular, the present invention provides oligomeric Aβ (1-40), Aβ (1-42), Aβ (3-38) and/or Aβ (11-38) as a plasma biomarker, which is suitable for a differential diagnosis of Alzheimer's disease, in particular in the early stages of the disease.

Therefore, in one embodiment, the invention is directed to the use of the method of determining the oligomeric state of amyloid p peptide for the diagnosis of Alzheimer's disease, such as the differential diagnosis of Alzheimer's disease, in particular in the early stages of the disease. Suitably, the early stage of Alzheimer's disease is Mild Cognitive impairment.

In a further embodiment, the invention is directed to the use of the oligomeric Aβ target peptides for the diagnosis of Alzheimer's diseases, such as the differential diagnosis of Alzheimer's disease, in particular in the early stages of the disease. Suitably, the early stage of Alzheimer's disease is Mild Cognitive impairment.

In particular, the oligomeric Aβ target peptide, which shall be used for diagnosis of Alzheimer's disease, is detected and quantified with a method according to the present invention.

In a further embodiment, the Aβ target peptide is Aβ (x-y), as hereinbefore defined, or a functional equivalent thereof.

The method of the invention also has industrial applicability to monitoring the efficacy of a given treatment of a neurodegenerative disorder, such as Alzheimer's disease. Thus, according to a further aspect of the invention there is provided a method of monitoring efficacy of a therapy in a subject having, suspected of having, or of being predisposed to, a neurodegenerative disorder, such as Alzheimer's disease, comprising determining the oligomeric state of a target amyloid β peptide (Abeta or Aβ) as defined herein in a biological sample from a test subject.

In one embodiment, the biological sample will be taken on two or more occasions from a test subject. In a further embodiment, the method additionally comprises comparing the level of the oligomeric state of a target amyloid β peptide (Abeta or Aβ) present in biological samples taken on two or more occasions from a test subject. In one embodiment, the method additionally comprises comparing the level of the oligomeric state of a target amyloid β peptide (Abeta or Aβ) present in a test sample with the amount present in one or more sample(s) taken from said subject prior to commencement of therapy, and/or one or more samples taken from said subject at an earlier stage of therapy. In one embodiment, the method additionally comprises comparing the level of the oligomeric state of a target amyloid β peptide (Abeta or Aβ) with one or more controls.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES OF THE INVENTION

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### 1. Materials and Methods

### 1.1 Patients and Healthy Controls

Patients with a clinical diagnosis of AD and healthy controls were recruited through a CRO (GALMED GmbH). In a prestudy examination the neuropsychological functions of all participants of the study were tested by several psychometric tests (DemTect, Mini-Mental-State Test, Clock-drawing test).

### DemTect Test

The DemTect scale is a brief screening for dementia comprising five short subtests (10-word list repetition, number transcoding, semantic word fluency task, backward digit span, delayed word list recall) (Kessler et al., 2000). The raw scores are transformed to give age- and education-independent scores, classified as ‘suspected dementia’ (score≦8), ‘mild cognitive impairment’ (score 9-12), and ‘appropriate for age’ (score 13-18).

### MMSE

The Mini-Mental State Examination (MMSE) or Folstein test is a brief 30-point questionnaire test that is used to assess cognition (see Table 1). It is commonly used in medicine to screen for dementia. In the time span of about 10 minutes it samples various functions including arithmetic, memory and orientation. It was introduced by Folstein et al., 1975, and is widely used with small modifications.

The MMSE includes simple questions and problems in a number of areas: the time and place of the test, repeating lists of words arithmetic, language use and comprehension, and basic motor skills. For example, one question asks to copy drawing of two pentagons (see next table). Any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing.

TABLE 1
Mini-Mental State Examination

| | | Max | |
|---|---|---|---|
| Section | Questions | Points | Score |
| 1) Orientation | a) Can you tell me today's | 5 | |
| | (date)/(month)/(year)? | | |
| | Which day is it today? | | |
| | Can you tell me which (season) it is? | | |
| | b) What town/city are we in? | 5 | |
| | What is the (county)! (country)? | | |
| | What (building) are we in and on | | |
| | what (floor) | | |
| 2) Registration | I should like to test your memory. | 3 | |
| | (name three common objects: “ball, car, man”) | | |
| | Can you repeat the words I said? | | |
| | (1 point per word) | | |
| | (repeat up to 6 trials until all three are | | |
| | remembered) | | |
| 3) Attention | a) From 100 keep subtracting 7 and give each | 5 | |
| and | answer. Stop after 5 answers. | | |
| Calculation | (93-86-79-72-65) | | |
| | Alternatively: | | |
| | b) Spell the word “World” backwards. | | |
| | (D_ L_R_O_ W) | | |
| 4) Recall | What were the three words I asked you to say | 3 | |
| | earlier? | | |
| | (skip this test if all of these objects were not | | |
| | remembered during the registration test | | |
| 5) Language Naming | Name the following objects (show a watch) and | 2 | |
| | (show a pencil) | | |
| Repeating | Repeat the following: “no ifs, ands or buts” | 1 | |
| 6) Reading | (show card or write: “Close your Eyes”) Read this | 1 | |
| | sentence and do what it says. | | |
| Writing | Now can you write a short sentence for me? | 1 | |
| 7) Three stage command | (present paper) | 3 | |
| | Take this paper in you left (or right) hand, fold it | | |
| | in half, and place it on the floor. | | |
| 8) Construction | Will you copy this drawing please? | 1 | |
| | 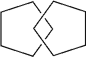 | | |
| Total score | | 30 | |

### Clock-Drawing Test

Scoring of the clocks was based on a modification of the scale used by Shulmann et al., 1986. All circles were pre-drawn and the instruction to subjects was to “set the time 10 after 11”. The scoring system (see Table 2) ranges in scores from 1 to 6 with higher scores reflecting a greater number of errors and more impairment. This scoring system is empirically derived and modified on the basis of clinical practice. Of necessity, it leaves considerable scope for individual judgment, but it is simple enough to have a high level of interrater reliability. Our study lends itself to the analysis of the three major components. These include cross-sectional comparisons of the clock-drawing test with other measures of cognitive function; a longitudinal description of the clock-drawing test over time, and the relationship between deterioration on the clock-drawing test and the decisions to institutionalize.

TABLE 2
Clock-drawing test

| | | |
|---|---|---|
| 1. | Perfect | [symbol] |
| 2. | Minor visuospatial errors | [symbol] |
| | Examples | |
| | Mildly impaired spacing of times | |
| | Draws times outside circle | |
| | Turns page while writing numbers so that some | |
| | numbers appear upside down | |
| | Draws in linies (spokes) to orient spacing | |
| 3. | Inaccurate representation of 10 after 11 when | [symbol] |
| | visuosptial organization is perfect or shows only minor | |
| | deviations | |
| | Examples | |
| | Minute hand points to 10 | |
| | Writes ‘10 after 11’ | |
| | Unable to make any denotation of time | |
| 4. | Moderate visuospatial disoganization of times such | [symbol] |
| | that accurate denotation of 10 after 11 is impossible | |
| | Example | |
| | Moderately poor spacing | |
| | Omits numbers | |
| | Perseveration - repeats circle or continues on | |
| | past 12 to 13, 14, 15, etc | |
| | Right-left reversal - numbers drawn counter | |
| | clockwise | |
| | Dysgraphia - unable to write numbers accurately | |
| 5. | Severe level of disorganization as described in 4 | [symbol] |
| 6. | No reasonable representation of a clock Exclude | [symbol] |
| | severe depression or other psychotic states | |
| | Examples | |
| | No attempt at all | |
| | No semblance of a clock at all | |
| | Writes a word or name | |

After Prestudy examination the study started 2 weeks later with blood withdrawal from all participants. Over one year with an interval of 3 months all participants had visited the center for the psychometric tests and blood samples withdrawal. The study was approved by the Ethics Committee of the “Ärztekammer Sachsen-Anhalt”. All patients (or their nearest relatives) and controls gave informed consent to participate in the study.

1.2 Blood samples

For the analysis of the Aβ 1-40 and/or Aβ 1-42 concentration in humans all of the following body fluids can be used: blood, cerebrospinal fluid, urine, lymph, saliva, sweat, pleura fluid, synovial fluid, aqueous fluid, tear fluid, bile and pancreas secretion.

The novel method was established with blood samples and can be further used for CSF, brain extract and urine samples, followed by all other human body fluids.

Blood samples for the determination of AD biomarkers were collected into three polypropylene tubes:
1. containing potassium-EDTA (Sarstedt Monovette, 02.1066.001) for EDTA plasma
2. containing Li-heparine (Sartstedt Monovette, 02.1065.001) for heparine plasma
3. blank (Sarstedt Monovette, 02.1063.001) for serum

All samples were collected by venous puncture or by repeated withdrawal out of an inserted forearm vein indwelling cannula. Blood was collected according to the time schedule (as described in section 1.1 above). It was centrifuged at 1550 g (3000 rpm) for 10 min at 4° C. to provide plasma. Plasma or serum was pipetted off, filled in one 5 ml polypropylene cryo-tube (Carl-Roth, E295.1) and stored frozen at −80° C. Samples were centrifuged within one hour after blood withdrawal. The appropriate labelling of the plasma or serum tubes according to the study protocol was duty of the CRO.

### 1.3 Laboratory Methods

Beside wild type Aβ 1-40 mutated variants can also be quantified by this method. The mutated variants comprise all amyloid beta peptides starting with amino acids Asp-Ala-Glu and ending with Gly-Val-Val. Mutated Aβ 1-40 examples:
Tottori, Flemish, Dutch, Italian, Arctic, Iowa (Irie et al., 2005)

The Aβ 1-40 assay can be also used for other familial Alzheimer's disease, which offer mutations outside the Aβ 1-40 sequence producing the wild type Aβ 1-40. Following familial Alzheimer's disease examples are also suitable for the assay:
Swedish, Austrian, French, German, Florida, London, Indiana, Australian (Irie et al., 2005)

Beside wild type Aβ 1-42 also mutated variants can be quantified by this method. The mutated variants comprise all amyloid beta peptides starting with amino acids Asp-Ala-Glu and ending with Val-Ile-Ala. Mutated Aβ 1-42 examples:
Tottori, Flemish, Dutch, Italian, Arctic, Iowa (Irie et al., 2005)

The Aβ 1-42 assay can be also used for other familial Alzheimer's disease, which offer mutations outside the Aβ 1-42 sequence producing the wild type Aβ 1-42. Following familial Alzheimer's disease examples are also suitable for the assay:
Swedish, Austrian, French, German, Florida, London, Indiana, Australian (Irie et al., 2005)

### Immunoprecipitation

EDTA plasma samples (containing 4 ml plasma) (heparin plasma, serum also possible) were thawed and aliquoted at 1 ml in 2 ml polypropylene tubes (Eppendorf, 0030120.094). One pill of protease inhibitor (Roche, Complete mini Protease inhibitor cocktail, 11836153001) was dissolved in 1 ml D-PBS (Invitrogen, 14190-094). 25 μl of the protease inhibitor solution was added to 1 ml EDTA plasma. All aliquots were frozen and stored again at −80° C., except one tube of each sample. These plasma tubes were spiked with 10 μl of 10% Tween-20. To each tube 2.5 μg anti-amyloid β (17-24) antibody 4G8 (Millipore, MAB1561), 2.5 μg anti-amyloid β (x-42) antibody 12F4 (Millipore, 05-831) and 2.5 μg anti-amyloid β (x-40) antibody 11A5-B10 (Millipore, 05-799) were added.

Other possible antibodies for immunoprecipitation are as defined hereinbefore. Beside these listed antibodies all other amyloid beta specific antibodies (monoclonal and polyclonal), which are suitable for immunoprecipitation can be used for this method (see also www.alzforum.org). Decisive for good capture efficiency is usage of two, three or more different antibodies with different epitopes. The usage of more than one antibody type for immunoprecipitation of, Aβ peptides offer cooperative binding effects (avidity), which yield tremendously higher capture efficiency (see FIG. 1).

All plasma tubes were incubated overnight at 4° C. in an overhead shaker. For immobilization of the amyloid β-antibody complex, 100 μl anti-mouse magnetic beads (Invitrogen, 112-02D) were used for a 1 ml plasma sample. Beside these special anti-mouse antibodies conjugated on magnetic beads all other anti-mouse antibodies or anti-host antibodies (host: origin of primary antibodies listed above) can be used. These antibodies can be immobilized on several matrices (column matrices and bead matrices) via different conjugation strategies, e.g. Biotin-Streptavidin interaction, tosyl-activated surface, epoxy-activated surface, amine-surface, carboxylic surface. Before usage, 100 μl beads were pipetted off from the original bottle into a 2 ml tube and washed 3-times with 1 ml PBS. After washing the beads were resuspended in 200 μl PBS. The plasma tubes were centrifuged for 30 sec at 2000×g. The supernatants were transferred into the tubes containing the anti-mouse magnetic beads. The tubes were incubated overnight at 4° C. in an overhead shaker.

On the next day the tubes were placed into a magnetic separator to allow the bead to be drawn to the tube wall. After about one minute the supernatant was carefully removed and the beads were washed twice with 500 μl D-PBS containing 10 mg/ml Cyclophilin 18 and 0.05% Tween-20.

Other washing buffers, which contain detergents or other additives preventing unspecific binding can be used for this step. Examples for washing buffers are:
PBS+0.05% Tween-20
TBS+0.05% Tween-20
Pierce ELISA Blocker (with Tween-20)

### Elution and Disaggregation of Captured Amyloid β

After the last wash step, the solution was drawn out, the tubes were taken from the magnetic separator and 100 μl 50% (v/v) Methanol/0.5% (v/v) formic acid were added to each tube and the beads were resuspended by slightly shaking. All tubes were incubated for 1 hour at room temperature. Afterwards the tubes were again placed in the magnetic separator and 40 μl eluate from each tube were mixed with 440 μl EIA buffer (dilution buffer of the IBL 1-40/42 (N) ELISA Kit). The pH of the diluted samples were adjusted with 16 μl 400 mM Na₂HPO₄, 400 mM KH₂PO₄ pH 8.0. From these samples the concentrations without disaggregation were determined. For disaggregation 50 μl eluate from each tube were transferred in new tubes and mixed with 20 μl 50% (v/v) Methanol/500 mM NaOH for every tube. The disaggregation was performed for 10 min at room temperature. Afterwards 40 μl from each disaggregation tube were mixed with 440 μl EIA buffer (dilution buffer of the IBL 1-40/42 (N) ELISA Kit). The pH of the diluted samples were adjusted with 10 μl 0.85% (v/v) H₃PO₄. From these samples the concentration after disaggregation were determined.

Beside the special ELISA dilution buffer from manufacturer IBL all other dilution buffer, which can prevent unspecific interaction with surfaces and capture antibodies, can be used for this step. Examples for dilution buffers are:
PBS+1% (w/v) BSA+0.05% Tween-20
TBS+1% (w/v) BSA+0.05% Tween-20
Pierce ELISA Blocker (with Tween-20)

### Quantification of the Eluted Amyloid β Peptides

The determination of the peptide concentration (with and without disaggregation, respectively) was performed using the IBL 1-40(N) ELISA Kit (IBL, JP27714) and IBL 1-42(N) ELISA Kit (IBL, JP27712).

Beside this special Aβ 1-40 ELISA all other commercially available, which are able to detect full length Aβ 1-40 can be used.

Examples for commercially ELISA-Kits:

| | |
|---|---|
| Human Abeta, aa 1-40 ELISA Kit | Invitrogen |
| Human Amyloid beta (Amyloid-b), | Wako Chemicals USA, Inc. |
| (aa 1-40 ELISA Kit) | |
| Amyloid Beta 1-40 ELISA Kit | The Genetics Company |

Self made Aβ 1-40 ELISA comprise of a pair of capture and detection antibody, which are specific for the complete N-terminus of Aβ 1-40 and the C-terminus ending at amino acid 40.

Possible N-terminal specific antibodies are:
3D6 (Elan Pharmaceuticals)
WO-2 (The Genetics Company)
1-40(N) detection antibody (IBL)
BAN50 (Takeda Chemicals Industries)

Possible C-terminal specific antibodies:
G2-10 (The Genetics Company)
11A5-B10 (Millipore)
1A10 (IBL)
Rabbit Anti-beta-Amyloid, aa 32-40 Polyclonal Antibody (GenScript Corporation)
EP1876Y, Epitope: x-40 (Novus Biologicals)

Such a self made full length Aβ 1-40 sandwich ELISA can comprise a first immobilized antibody recognizing specifically the C-terminus of Aβ 1-40 and a second labeled detection antibody recognizing specifically the complete N-terminus of Aβ 1-40. A full length Aβ 1-40 sandwich ELISA can also comprise a first immobilized antibody recognizing specifically the complete N-terminus of Aβ 1-40 and a second labeled detection antibody recognizing specifically the C-terminus of Aβ 1-40, this type of Aβ 1-40 sandwich ELISA is particularly envisaged.

Beside this special Aβ 1-42 ELISA all other commercially available, which are able to detect full length Aβ 1-42 can be used.

Examples for commercially ELISA-Kits:

| | |
|---|---|
| Human Abeta, aa 1-40 ELISA Kit | Invitrogen |
| Human Amyloid beta (Amyloid-b), | Wako Chemicals USA, Inc. |
| (aa 1-42 ELISA Kit) | |
| Amyloid Beta 1-42 ELISA Kit | The Genetics Company |
| beta-Amyloid 1-42 ELISA Kit (SIGNET) | Covance |
| INNOTEST ® β-AMYLOID (1-42) | Innogenetics |

Self made Aβ 1-40 ELISA comprise of a pair of capture and detection antibody, which are specific for the complete N-terminus of Aβ 1-42 and the C-terminus ending at amino acid 40.

Possible N-terminal specific antibodies are:
3D6 (Elan Pharmaceuticals)
WO-2 (The Genetics Company)
1-40(N) detection antibody (IBL)
BAN50 (Takeda Chemicals Industries)

Possible C-terminal specific antibodies:
G2-11 (The Genetics Company)
16C11 (Santa Cruz Biotechnology)
21F12 (Elan Pharmaceuticals, Innogenetics)
BC05 (Takeda Chemicals Industries)

Such a self made full length Aβ 1-42 sandwich ELISA can comprise a first immobilized antibody recognizing specifically the C-terminus of Aβ 1-42 and a second labeled detection antibody recognizing specifically the complete N-terminus of Aβ 1-42. A full length Aβ 1-42 sandwich ELISA can also comprise a first immobilized antibody recognizing specifically the complete N-terminus of Aβ 1-42 and a second labeled detection antibody recognizing specifically the C-terminus of Aβ 1-42, this type of Aβ 1-42 sandwich ELISA is particularly envisaged.

The diluted samples (with and without disaggregation, respectively) were applied to the ELISA plate (100 μl per well, repeat determination). The ELISA standard were taken from the kit, dissolved and diluted according to the manufacture instruction protocol. After application of all samples and concentration standards the ELISA plate was incubated for 18 h at 4° C. On the following day, the ELISA was developed according to the manufacturers instruction protocol.

After stopping the colorimetric reaction the absorbance in each well was determined at 450 nm corrected by absorbance at 550 nm using a plate reader (TECAN Sunrise).

The determination of the standard curve was completed by plotting of the corrected absorbance at 450 nm versus the corresponding standard peptide concentration. The curve was fitted with the four-parameter equation (Equ. 1) using Origin 7.0 (Microcal).

$$y = \frac{A 1 - A 2}{1 + {( \frac{x}{{x}_{0}} )}^{p}} + A 2 , \qquad Equ . 1$$

wherein y represents the measured absorbance and x represents the corresponding concentration

The calculation of the Aβ (1-40) and Aβ (1-42) concentrations on ELISA of each sample was completed based on the according absorbance value using Equ. 2.

$$x = {x}_{0} \cdot \sqrt[p]{\frac{A 1 - y}{y - A 2}} \qquad Equ . 2$$

To determine the concentration in the plasma sample, determined without disaggregation, the calculated concentration was corrected by the EIA buffer dilution (including pH adjustment), factor 12.4, and the concentration effect (1 ml to 100 μl) of the immunoprecipitation by factor 0.1. To determine the concentration in the plasma sample, determined with disaggregation, the calculated concentration was corrected by the EIA buffer dilution (including pH adjustment), factor 12.25, the dilution by adding 20 μl 50% (v/v) Methanol/500 mM NaOH to the eluted sample, factor 1.4, and the concentration effect (1 ml to 100 μl) of the immunoprecipitation by factor 0.1. The determined plasma Aβ (1-40/42) concentrations (with and without disaggregation, respectively) were denoted in pg/ml.

### Calculation and Statistical Analysis

For every plasma sample four parameters were determined:
1. Aβ (1-40) concentration (with disaggregation)
2. Aβ (1-40) concentration (without disaggregation)
3. Aβ (1-42) concentration (with disaggregation)
4. Aβ (1-42) concentration (without disaggregation)

From these data the ratio values for:

Oligomeric state Aβ (1-40)=Aβ 1-40 (with disaggregation)/Aβ 1-40 (without disaggregation)
Oligomeric state Aβ (1-42)=Aβ 1-42 (with disaggregation)/Aβ 1-42 (without disaggregation)
were calculated.

The association of plasma oligomeric state of Aβ (1-40) and Aβ (1-42) was examined with the existence of a positive clinical diagnosis of Alzheimer's disease using the Student's t-Test.

### 2. Results

### 2.1 Demographic Characteristics

Overall 45 persons have participated in the study, 30 healthy controls and 15 AD patients. To observe possible influences of age on plasma Aβ, control persons were selected over a wide range of age and subclassified into three groups, Group I contains age of 18 to 30, Group II from 31 to 45 and Group III from 46 to 65. The demographic characteristics are shown in Table 3.

TABLE 3
Demographic Characteristics

| | Healthy controls | | | |
|---|---|---|---|---|
| | Group I | Group II | Group III | |
| | (18-30) | (31-45) | (46-65) | AD patients |
| No. | 10 | 10 | 10 | 15 |
| Age at | 25.8 ± 2.9 | 38.4 ± 4.7 | 54 ± 6.9 | 79.13 ± 7.09 |
| baseline | | | | |
| (mean ± | | | | |
| SDEV), | | | | |
| Height, cm | 175.5 ± 11.6 | 175.1 ± 7.2 | 167.5 ± 10.9 | 168.4 ± 10.34 |
| (mean ± | | | | |
| SDEV) | | | | |
| Weight, kg | 71.33 ± 11.8 | 71.36 ± 13.5 | 75.81 ± 13.3 | 72.00 ± 12.31 |
| (mean ± | | | | |
| SDEV) | | | | |
| Sex (% | 50 | 50 | 50 | 40 |
| women) | | | | |

2.2 Psychometric tests

For evaluation of the neuropsychological functions all participants have performed the DemTect, Mini-Mental-State Test and Clock-Drawing test. These tests have been made in prestudy, 3 month, 6 month, 9 month and 12 month after the start of the study.

### DemTect Test

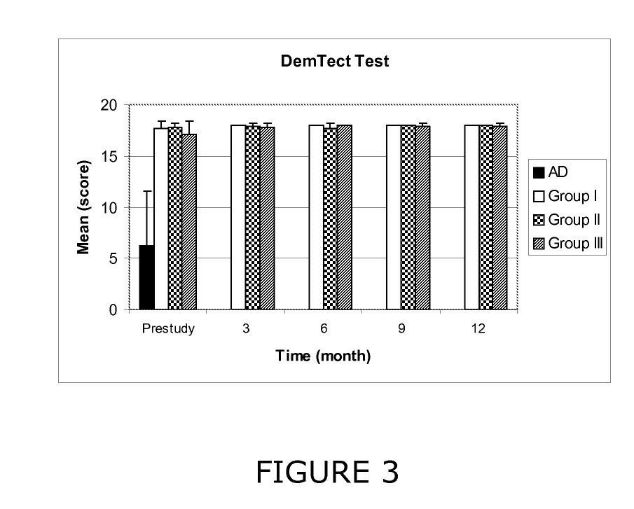

The raw scores are transformed to give age- and education-independent scores, classified as ‘suspected dementia’ (score≦8), ‘mild cognitive impairment’ (score 9-12), and ‘appropriate for age’ (score 13-18). The test results for all visits are shown in FIG. 3. The results from FIG. 3 demonstrate that there are clear differences between the three groups of healthy subjects compared with the patients.

### Mini-Mental-State Test

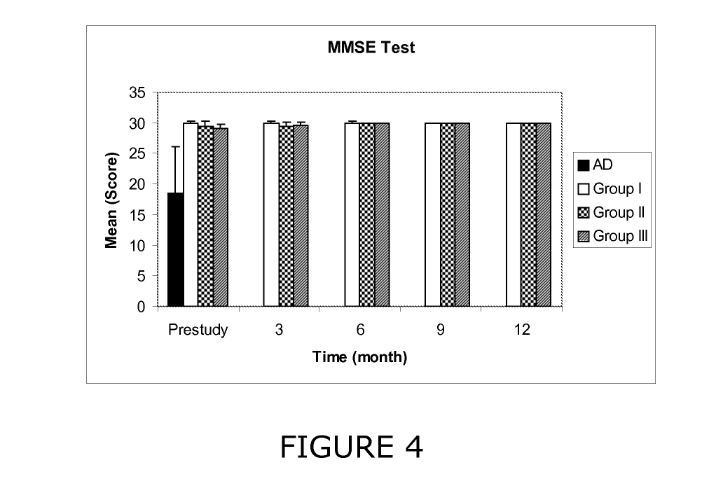

Any score over 27 (out of 30) is effectively normal. Below this, 20-26 indicates mild dementia; 10-19 moderate dementia, and below 10 severe dementia. The normal value is also corrected for degree of schooling and age. Low to very low scores correlate closely with the presence of dementia, although other mental disorders can also lead to abnormal findings on MMST testing. The test results are shown in FIG. 4. The results from FIG. 4 demonstrate that there are clear differences between the three groups of healthy subjects compared with the patients.

### Clock-Drawing Test

The scoring system ranges in scores from 1 to 6 with higher scores reflecting a greater number of errors and more impairment. This scoring system is empirically derived and modified on the basis of clinical practice. Of necessity, it leaves considerable scope for individual judgment, but it is simple enough to have a high level of interrater reliability.

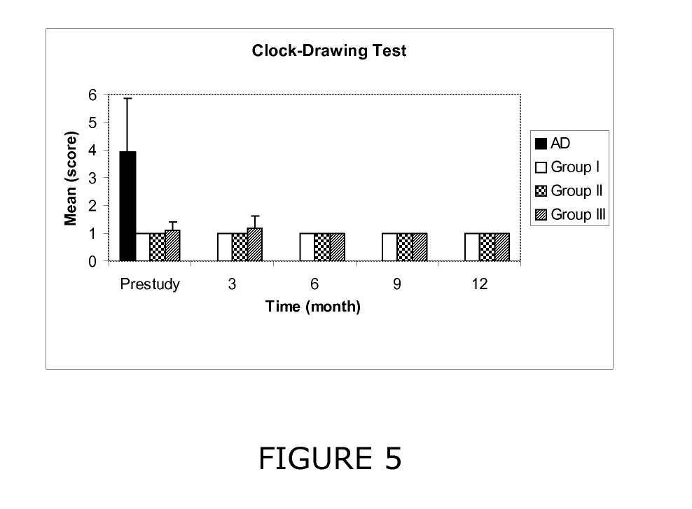

Our study lends itself to the analysis of the three major components. These include cross-sectional comparisons of the clock-drawing test with other measures of cognitive function; a longitudinal description of the clock-drawing test over time, and the relationship between deterioration on the clock-drawing test and the decisions to institutionalize. The test results are shown in FIG. 5. The results from FIG. 5 demonstrate that there are clear differences between the three groups of healthy subjects compared with the patients.

### 2.3 Plasma Oligomeric State of Aβ (1-40) and Aβ (1-42)

The Aβ (1-40/42) concentrations (with and without disaggregation, respectively) were determined in EDTA plasma of the T0+9 month series. Because of two serious adverse events, AD patient Nr. 34 and 35 were late, only 13 AD samples were unhanded by the CRO for investigations. Further samples of T0+9 series were used to optimize and establish the new immunoprecipitation method. Overall, the final optimized method was tested with 11 AD samples and 26 control samples. The determined concentrations are shown in Table 4.

TABLE 4
Plasma oligomeric state of Aβ (1-40) and Aβ (1-42) (T0 + 9 month series)

| AD Group | | | | | | | | | | | |
|---|---|---|---|---|---|---|---|---|---|---|---|
| | Oligomeric state | | Control Group 18-30 | | | Control Group 31-45 | | | Control Group 46-65 | | |
| | Abeta | | | Oligomeric state | | | Oligomeric state | | | Oligomeric state | |
| Subject | 1-40 | Abeta 1-42 | Subject | Abeta 1-40 | Abeta 1-42 | Subject | Abeta 1-40 | Abeta 1-42 | Subject | Abeta 1-40 | Abeta 1-42 |
| Nr. 10 | 1.259 | 1.075 | Nr. 09 | 1.176 | | Nr. 13 | 1.532 | 1.281 | Nr. 02 | 1.297 | 1.229 |
| Nr. 11 | 1.060 | 0.846 | Nr. 27 | 1.052 | 1.308 | Nr. 15 | 1.272 | 1.242 | Nr. 08 | 1.149 | |
| Nr. 14 | 1.065 | 1.220 | Nr. 32 | 1.562 | 1.277 | Nr. 17 | 1.173 | 1.190 | Nr. 12 | 1.112 | 1.553 |
| Nr. 16 | 1.090 | 1.276 | Nr. 36 | 1.591 | 1.246 | Nr. 18 | 1.372 | 1.209 | Nr. 19 | 1.374 | 1.250 |
| Nr. 20 | 1.086 | 1.055 | Nr. 37 | 1.206 | 1.230 | Nr. 25 | 1.030 | 1.178 | Nr. 21 | 1.242 | 1.203 |
| Nr. 22 | 1.251 | 1.084 | Nr. 40 | 1.287 | 1.138 | Nr. 28 | 1.409 | 1.264 | Nr. 23 | 1.257 | 1.108 |
| Nr. 26 | 1.152 | 1.154 | Nr. 41 | 1.151 | 1.810 | Nr. 29 | 1.580 | 1.383 | Nr. 24 | 1.250 | 1.211 |
| Nr. 30 | 1.114 | 1.055 | Nr. 42 | 1.259 | 1.091 | Nr. 31 | 1.376 | 1.254 | Nr. 33 | 1.662 | 1.171 |
| Nr. 39 | 1.233 | 1.037 | Nr. 44 | 1.217 | 1.084 | Nr. 38 | 1.559 | 1.353 | | | |
| Nr. 43 | | 0.814 | | | | | | | | | |
| Nr. 45 | 1.166 | | | | | | | | | | |
| Mean | 1.148 | 1.062 | Mean | 1.278 | 1.273 | Mean | 1.367 | 1.262 | Mean | 1.293 | 1.246 |
| SEM | 0.024 | 0.046 | SEM | 0.061 | 0.082 | SEM | 0.061 | 0.023 | SEM | 0.060 | 0.054 |

| | | | | | | |
|---|---|---|---|---|---|---|
| T-Test AD Group vs. Control | 0.054 | 0.031 | 0.003 | 0.002 | 0.028 | 0.020 |
| group | | | | | | |

The mean values and the standard error of mean for all four groups were calculated.
The T-test has compared the AD group with each single control group.
The values for the oligomeric state in table 4 were calculated according the methods of the present invention and represent the ratio c_{d}/cₐ.

Concerning the oligomeric state of Aβ (1-42) for all control groups, a significant increased value was obtained compared with the AD group. The same result was obtained by comparison of oligomeric state of Aβ (1-40). Only group 18-30 has curtly missed the significance.

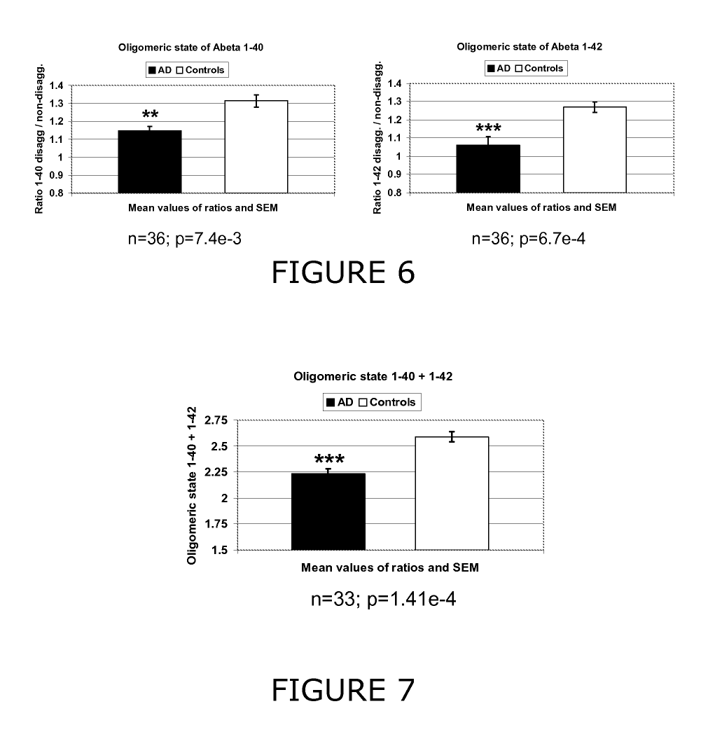

The oligomeric state of Aβ (1-40) and Aβ (1-42) of all control samples against all samples of the AD group (FIG. 6) was also evaluated. The oligomeric state of Aβ (1-40) and Aβ (1-42), respectively, were significantly decreased in AD patients compared with healthy controls. For the oligomeric state of Aβ (1-40) and Aβ (1-42) p-values of 0.0074 and 0.00067, respectively, were obtained.

The used method cannot determine the amount of Aβ (1-40) or Aβ (1-42) homo-oligomers in the sample, it displays the amount of Aβ (1-40) and Aβ (1-42) peptides within soluble aggregates compared with monomeric Aβ (1-40) and Aβ (1-42) in the sample. Because of this fact the summation of the values for Aβ (1-40) and Aβ (1-42) can reflect the overall amount of Aβ oligomers in plasma of AD patients and healthy controls (FIG. 7).

The summation of oligomeric state values has further improved the p-value of the T-test, although the sample quantity was less compared with single evaluation for Aβ (1-40) and Aβ (1-42), respectively.

### 3. Discussion

The results presented herein show that a decrease of the oligomeric state of Aβ 1-40 and Aβ 1-42 were associated with a positive clinical diagnosis of Alzheimer's Disease. The summation of both oligomeric states (Aβ 1-40+Aβ 1-42) improves the significance (p=1.41e−4). Until now, there exist only a few comparable studies in the literature. In one study they could show that the plasma level of proto-fibrillar Aβ42 declined over the follow-up in those who had developed mild AD by the second assessment (Schupf et al., 2008), what supports our data. However, the plasma levels of protofibrillar Aβ42 were only detectable in 34% of all participants (1125 elderly persons). This fact constrains the usability of this assay, which uses a monoclonal antibody (clone 13C3) generated by immunization of mice with a fibrillar form of Aβ42. The characterization of the 13C3 antibody offers a good affinity to protofibrillar Aβ42, however also to monomeric Aβ42 (Schupf et al., 2008; supporting information), which can falsify the determined protofibrillar Aβ42 level. Therefore the usage of such assay systems based on oligomer or protofibrillar specific antibodies is hampered, if the detection antibody is not exclusively specific for higher molecular aggregates of Amyloid β. In another study (Xia et al., 2009) a sandwich ELISA used the same antibody for capture and detection for detection of oligomeric Aβ. Thus a detection is only possible if the Aβ assembly contains at least two exposed copies of the same epitope that is accessible by the identical capturing and detection antibody (El-Agnaf et al., 2000; Howlett et al., 1999). Xia and co-workers found an increased plasma level of oligomeric Aβ with a p-value<0.05, which is contradictory to the findings presented herein. However also in this study only in 30% of healthy controls and in 52% of AD patients oligomeric Aβ were detectable, which constrains the usability also of this assay.

Both studies show the same problem, the aggregated amyloid β is not detectable in all samples. It is possible that the issue is caused by an inefficient and not reliable recovery rate of amyloid beta by a simple sandwich ELISA. In the invention the bivalent capture system ensures a complete recovery of all Aβ molecules from the sample, which makes the present assay more reliable.

A very recent publication offers a method that uses also a indirect quantification of oligomeric amyloid β (Englund et al., 2009). This study analyzed CSF samples and quantified the Aβ (1-42) level under denaturing and non-denaturing conditions and calculated the Aβ42 oligomeric ratio CSF samples. They found an increased ratio in samples of AD and MCI compared with healthy controls. However, this assay is constrained by the usage of different methods for quantification of denatured and non-denatured Aβ42. For non-denatured condition the Aβ42 concentration is determined by a normal sandwich ELISA. As described above, such a simple sandwich ELISA could have problems with the recovery rate. For denatured conditions the Aβ42 concentration is determined by SDS-PAGE followed by Western Blot analysis. A critical issue of this method is the fact that Aβ (1-42) assemblies cannot completely disaggregate to monomer by 2% SDS. Our experiences show also trimer and tetramer species of Aβ (1-42) in SDS-PAGE. Against this background a correct quantification of Aβ (1-42) monomers is very doubtful. Furthermore this fact makes a comparison with ELISA determined concentration and subsequent the calculation of a ratio of both values very defective.

Until now, all published methods for quantification of amyloid β oligomers or protofibrils exhibit critical issues, they are only constricted applicable for analyzing human plasma and CSF, respectively. The invention overcomes these issues and show reliable detection of Aβ aggregates in human plasma.

### REFERENCES

Blennow K, de Leon M J, Zetterberg H. Alzheimer's disease. Lancet. 2006 Jul. 29; 368(9533):387-403
Ferri C P, Prince M, Brayne C, Brodaty H, Fratiglioni L, Ganguli M, Hall K, Hasegawa K, Hendrie H, Huang Y, Jorm A, Mathers C, Menezes P R, Rimmer E, Scazufca M
Global prevalence of dementia: a Delphi consensus study. Lancet. 2005 Dec. 17; 366(9503):2112-7
Knopman D S, DeKosky S T, Cummings J L, Chui H, Corey-Bloom J, Relkin N, Small G W, Miller B, Stevens J C. Practice parameter: diagnosis of dementia (an evidence-based review). Report of the Quality Standards Subcommittee of the American Academy of Neurology. Neurology. 2001 May 8; 56(9):1143-53
Waldemar G, Dubois B, Emre M, Georges J, McKeith I G, Rossor M, Scheltens P, Tariska P, Winblad B; EFNS. Recommendations for the diagnosis and management of Alzheimer's disease and other disorders associated with dementia: EFNS guideline.
Eur J Neurol. 2007 January; 14 (1):e1-26.
Dubois B, Feldman H H, Jacova C, Dekosky S T, Barberger-Gateau P, Cummings J, Delacourte A, Galasko D, Gauthier S, Jicha G, Meguro K, O'brien J, Pasquier F, Robert P, Rossor M, Salloway S, Stern Y, Visser P J, Scheltens P. Research criteria for the diagnosis of Alzheimer's disease: revising the NINCDS-ADRDA criteria. Lancet Neurol. 2007 August; 6(8):734-46.
Jellinger K A. Alzheimer's disease. In: Gilman S, editor. Neurobiology of Disease. Amsterdam: Elsevier Academic Press; 2007. p. 69-82
Petersen R C, Smith G E, Waring S C, Ivnik R J, Tangalos E G, Kokmen E. Mild cognitive impairment: clinical characterization and outcome. Arch Neurol. 1999 March; 56(3):303-8
Chertkow H, Massoud F, Nasreddine Z, Belleville S, Joanette Y, Bocti C, Drolet V, Kirk J, Freedman M, Bergman H. Diagnosis and treatment of dementia: 3. Mild cognitive impairment and cognitive impairment without dementia. CMAJ. 2008 May 6; 178(10):1273-85
Scheff S W, Price D A, Schmitt F A, Mufson E J. Hippocampal synaptic loss in early Alzheimer's disease and mild cognitive impairment. Neurobiol Aging. 2006 October; 27(10):1372-84
Markesbery W R, Schmitt F A, Kryscio R J, Davis D G, Smith C D, Wekstein D R. Neuropathologic substrate of mild cognitive impairment. Arch Neurol. 2006 January; 63(1): 38-46.
Bouwman F H, Schoonenboom S N, van der Flier W M, van Elk E J, Kok A, Barkhof F, Blankenstein M A, Scheltens P. CSF biomarkers and medial temporal lobe atrophy predict dementia in mild cognitive impairment. Neurobiol Aging. 2007 July; 28(7): 1070-4
Saito Y, Murayama S, Neuropathology of mild cognitive impairment. Neuropathology. 2007 December; 27(6):578-84
Jicha G A, Parisi J E, Dickson D W, Johnson K, Cha R, Ivnik R J, Tangalos E G, Boeve B F, Knopman D S, Braak H, Petersen R C. Neuropathologic outcome of mild cognitive impairment following progression to clinical dementia. Arch Neurol. 2006 May; 63(5):674-81
Petersen R C, Parisi J E, Dickson D W, Johnson K A, Knopman D S, Boeve B F, Jicha G A, Ivnik R J, Smith G E, Tangalos E G, Braak H, Kokmen E. Neuropathologic features of amnestic mild cognitive impairment. Arch Neurol. 2006 May; 63(5):665-72
Gauthier S, Reisberg B, Zaudig M, Petersen R C, Ritchie K, Broich K, Belleville S, Brodaty H, Bennett D, Chertkow H, Cummings J L, de Leon M, Feldman H, Ganguli M, Hampel H, Scheltens P, Tierney M C, Whitehouse P, Winblad B; International Psychogeriatric Association Expert Conference on mild cognitive impairment. Mild cognitive impairment. Lancet. 2006 Apr. 15; 367(9518):1262-70
Fischer P, Jungwirth S, Zehetmayer S, Weissgram S, Hoenigschnabl S, Gelpi E, Krampla W, Tragl K H. Conversion from subtypes of mild cognitive impairment to Alzheimer dementia. Neurology. 2007 Jan. 23; 68(4):288-91
Devanand D P, Pradhaban G, Liu X, Khandji A, De Santi S, Segal S, Rusinek H, Pelton G H, Honig L S, Mayeux R, Stern Y, Tabert M H, de Leon M J. Hippocampal and entorhinal atrophy in mild cognitive impairment: prediction of Alzheimer disease. Neurology. 2007 Mar. 13; 68(11):828-36
Rossi R, Geroldi C, Bresciani L, Testa C, Binetti G, Zanetti O, Frisoni G B. Clinical and neuropsychological features associated with structural imaging patterns in patients with mild cognitive impairment. Dement Geriatr Cogn Disord. 2007; 23(3):175-83
Whitwell J L, Petersen R C, Negash S, Weigand S D, Kantarci K, Ivnik R J, Knopman D S, Boeve B F, Smith G E, Jack C R Jr. Patterns of atrophy differ among specific subtypes of mild cognitive impairment. Arch Neurol. 2007 August; 64(8):1130-8
Panza F, Capurso C, D'Introno A, Colacicco A M, Capurso A, Solfrizzi V. Heterogeneity of mild cognitive impairment and other predementia syndromes in progression to dementia. Neurobiol Aging. 2007 October; 28(10):1631-2; discussion 1633-4
Hyman S E. Can neuroscience be integrated into the DSM-V? Nat Rev Neurosci. 2007 September; 8(9):725-32
Blennow K. Cerebrospinal fluid protein biomarkers for Alzheimer's disease. NeuroRx. 2004 April; 1(2):213-25
Blennow K. CSF biomarkers for Alzheimer's disease: use in early diagnosis and evaluation of drug treatment. Expert Rev Mol Diagn. 2005 September; 5(5):661-72
Hampel H, Buerger K. Biomarkers in blood and cerebrospinal fluid. In: Herholz K, Morris C, Perani D, editors. The Dementias: Early Diagnosis and Evaluation. New York: Taylor & Francis; 2006. p. 73-107
Lewczuk P, Kornhuber J, Wiltfang J. The German Competence Net Dementias: standard operating procedures for the neurochemical dementia diagnostics. J Neural Transm. 2006 August; 113(8): 1075-80
Irizarry M C. Biomarkers of Alzheimer disease in plasma. NeuroRx. 2004 April; 1(2):226-34
Barnes J, Foster J, Fox N C. Structural magnetic resonance imaging-derived biomarkers for Alzheimer's disease. Biomarkers Med. 2007; 1: 79-92
Vemuri P, Gunter J L, Senjern M L, Whitwell J L, Kantarci K, Knopman D S, Boeve B F, Petersen R C, Jack C R Jr. Alzheimer's disease diagnosis in individual subjects using structural MR images: validation studies. Neuroimage. 2008 Feb. 1; 39(3):1186-97
Barkhof F, Polvikoski T M, van Straaten E C, Kalaria R N, Sulkava R, Aronen H J, Niinistö L, Rastas S, Oinas M, Scheltens P, Erkinjuntti T. The significance of medial temporal lobe atrophy: a postmortem MRI study in the very old. Neurology. 2007 Oct. 9; 69(15):1521-7
Mevel K, Desgranges B, Baron J C, Landeau B, De la Sayette V, Viader F, Eustache F, Chételat G. Detecting hippocampal hypometabolism in Mild Cognitive Impairment using automatic voxel-based approaches. Neuroimage. 2007 Aug. 1; 37(1):18-25
Kemppainen N M, Aalto S, Wilson I A, Någren K, Helin S, Brück A, Oikonen V, Kailajärvi M, Scheinin M, Viitanen M, Parkkola R, Rinne J O. PET amyloid ligand [11C]PIB uptake is increased in mild cognitive impairment. Neurology. 2007 May 8; 68(19):1603-6
Klunk W E, Engler H, Nordberg A, Wang Y, Blomqvist G, Holt D P, Bergström M, Savitcheva I, Huang G F, Estrada S, Ausén B, Debnath M L, Barletta J, Price J C, Sandell J, Lopresti B J, Wall A, Koivisto P, Antoni G, Mathis C A, Långström B. Imaging brain amyloid in Alzheimer's disease with Pittsburgh Compound-B. Ann Neurol. 2004 March; 55(3):306-19
Rowe C C, Ng S, Ackermann U, Gong S J, Pike K, Savage G, Cowie T F, Dickinson K L, Maruff P, Darby D, Smith C, Woodward M, Merory J, Tochon-Danguy H, O'Keefe G, Klunk W E, Mathis C A, Price J C, Masters C L, Villemagne V L. Imaging beta-amyloid burden in aging and dementia. Neurology. 2007 May 15; 68(20):1718-25
Pike K E, Savage G, Villemagne V L, Ng S, Moss S A, Maruff P, Mathis C A, Klunk W E, Masters C L, Rowe C C. Beta-amyloid imaging and memory in non-demented individuals: evidence for preclinical Alzheimer's disease. Brain. 2007 November; 130 (Pt 11):2837-44
Huang C, Eidelberg D, Habeck C, Moeller J, Svensson L, Tarabula T, Julin P. Imaging markers of mild cognitive impairment: multivariate analysis of CBF SPECT. Neurobiol Aging. 2007 July; 28(7):1062-9
Kantarci K, Weigand S D, Petersen R C, Boeve B F, Knopman D S, Gunter J, Reyes D, Shiung M, O'Brien P C, Smith G E, Ivnik R J, Tangalos E G, Jack C R Jr. Longitudinal 1H MRS changes in mild cognitive impairment and Alzheimer's disease. Neurobiol Aging. 2007 September; 28(9):1330-9
Petrella J R, Wang L, Krishnan S, Slavin M J, Prince S E, Tran T T, Doraiswamy P M. Cortical deactivation in mild cognitive impairment: high-field-strength functional MR imaging. Radiology. 2007 October; 245(1):224-35
Hämäläinen A, Tervo S, Grau-Olivares M, Niskanen E, Pennanen C, Huuskonen J, Kivipelto M, Hänninen T, Tapiola M, Vanhanen M, Hallikainen M, Helkala E L, Nissinen A, Vanninen R, Soininen H. Voxel-based morphometry to detect brain atrophy in progressive mild cognitive impairment. Neuroimage. 2007 Oct. 1; 37(4):1122-31
Kircher T T, Weis S, Freymann K, Erb M, Jessen F, Grodd W, Heun R, Leube D T. Hippocampal activation in patients with mild cognitive impairment is necessary for successful memory encoding. J Neurol Neurosurg Psychiatry. 2007 August; 78(8): 812-8
Kropholler M A, Boellaard R, van Berckel B N, Schuitemaker A, Kloet R W, Lubberink M J, Jonker C, Scheltens P, Lammertsma A A. Evaluation of reference regions for (R)-[(11)C]PK11195 studies in Alzheimer's disease and mild cognitive impairment. J Cereb Blood Flow Metab. 2007 December; 27(12):1965-74
Attems J. Sporadic cerebral amyloid angiopathy: pathology, clinical implications, and possible pathomechanisms. Acta Neuropathol. 2005 October; 110(4):345-59
Blennow K, Hampel H. CSF markers for incipient Alzheimer's disease. Lancet Neurol. 2003 October; 2(10):605-13

Hansson O, Zetterberg H, Buchhave P, Londos E, Blennow K, Minthon L. Association between CSF biomarkers and incipient Alzheimer's disease in patients with mild cognitive impairment: a follow-up study. Lancet Neurol. 2006 March; 5(3):228-34
Hansson O, Zetterberg H, Buchhave P, Andreasson U, Londos E, Minthon L, Blennow K. Prediction of Alzheimer's disease using the CSF Abeta42/Abeta40 ratio in patients with mild cognitive impairment. Dement Geriatr Cogn Disord. 2007; 23(5):316-20
Fagan A M, Mintun M A, Mach R H, Lee S Y, Dence C S, Shah A R, LaRossa G N, Spinner M L, Klunk W E, Mathis C A, DeKosky S T, Morris J C, Holtzman D M. Inverse relation between in vivo amyloid imaging load and cerebrospinal fluid Abeta42 in humans. Ann Neurol. 2006 March; 59(3):512-9
Prince J A, Zetterberg H, Andreasen N, Marcusson J, Blennow K. APOE epsilon4 allele is associated with reduced cerebrospinal fluid levels of Abeta42. Neurology. 2004 Jun. 8; 62(11):2116-8
Strozyk D, Blennow K, White L R, Launer L J. CSF Abeta 42 levels correlate with amyloid-neuropathology in a population-based autopsy study. Neurology. 2003 Feb. 25; 60(4):652-6
Walsh D M, Klyubin I, Shankar G M, Townsend M, Fadeeva J V, Betts V, Podlisny M B, Cleary J P, Ashe K H, Rowan M J, Selkoe D J. The role of cell-derived oligomers of Abeta in Alzheimer's disease and avenues for therapeutic intervention. Biochem Soc Trans. 2005 November; 33 (Pt 5):1087-90
Ewers M, Buerger K, Teipel S J, Scheltens P, Schröder J, Zinkowski R P, Bouwman F H, Schönknecht P, Schoonenboom N S, Andreasen N, Wallin A, DeBernardis J F, Kerkman D J, Heindl B, Blennow K, Hampel H. Multicenter assessment of CSF-phosphorylated tau for the prediction of conversion of MCI. Neurology. 2007 Dec. 11; 69(24):2205-12
Fagan A M, Roe C M, Xiong C, Mintun M A, Morris J C, Holtzman D M. Cerebrospinal fluid tau/beta-amyloid(42) ratio as a prediction of cognitive decline in nondemented older adults. Arch Neurol. 2007 March; 64(3):343-9.
Gustafson D R, Skoog I, Rosengren L, Zetterberg H, Blennow K. Cerebrospinal fluid beta-amyloid 1-42 concentration may predict cognitive decline in older women. J Neurol Neurosurg Psychiatry. 2007 May; 78(5):461-4
Li G, Sokal I, Quinn J F, Leverenz J B, Brodey M, Schellenberg G D, Kaye J A, Raskind M A, Zhang J, Peskind E R, Montine T J. CSF tau/Abeta42 ratio for increased risk of mild cognitive impairment: a follow-up study. Neurology. 2007 Aug. 14; 69(7):631-9
Stomrud E, Hansson O, Blennow K, Minthon L, Londos E. Cerebrospinal fluid biomarkers predict decline in subjective cognitive function over 3 years in healthy elderly. Dement Geriatr Cogn Disord. 2007; 24(2):118-24
Hampel H, Teipel S J, Fuchsberger T, Andreasen N, Wiltfang J, Otto M, Shen Y, Dodel R, Du Y, Farlow M, Möller H J, Blennow K, Buerger K. Value of CSF beta-amyloid 1-42 and tau as predictors of Alzheimer's disease in patients with mild cognitive impairment. Mol. Psychiatry. 2004 July; 9(7):705-10
Maccioni R B, Lavados M, Guillón M, Mujica C, Bosch R, Farías G, Fuentes P. Anomalously phosphorylated tau and Abeta fragments in the CSF correlates with cognitive impairment in MCI subjects. Neurobiol Aging. 2006 February; 27(2):237-44
Schönknecht P, Pantel J, Kaiser E, Thomann P, Schröder J. Increased tau protein differentiates mild cognitive impairment from geriatric depression and predicts conversion to dementia. Neurosci Lett. 2007 Apr. 6; 416(1):39-42
Clark C M, Xie S, Chittams J, Ewbank D, Peskind E, Galasko D, Morris J C, McKeel D W Jr, Farlow M, Weitlauf S L, Quinn J, Kaye J, Knopman D, Arai H, Doody R S, DeCarli C, Leight S, Lee V M, Trojanowski J Q. Cerebrospinal fluid tau and beta-amyloid: how well do these biomarkers reflect autopsy-confirmed dementia diagnoses? Arch Neurol. 2003 December; 60(12):1696-702
Buerger K, Ewers M, Pirttilä T, Zinkowski R, Alafuzoff I, Teipel S J, DeBernardis J, Kerkman D, McCulloch C, Soininen H, Hampel H. CSF phosphorylated tau protein correlates with neocortical neurofibrillary pathology in Alzheimer's disease. Brain. 2006 November; 129 (Pt 11):3035-41
Engelborghs S, Sleegers K, Cras P, Brouwers N, Serneels S, De Leenheir E, Martin J J, Vanmechelen E, Van Broeckhoven C, De Deyn P P. No association of CSF biomarkers with APOEepsilon4, plaque and tangle burden in definite Alzheimer's disease. Brain. 2007 September; 130 (Pt 9):2320-6

Buerger K, Alafuzoff I, Ewers M, Pirttilä T, Zinkowski R, Hampel H. No correlation between CSF tau protein phosphorylated at threonine 181 with neocortical neurofibrillary pathology in Alzheimer's disease. Brain. 2007 October; 130 (Pt 10):e82

Wiltfang J, Esselmann H, Bibl M, Hüll M, Hampel H, Kessler H, Frölich L, Schröder J, Peters O, Jessen F, Luckhaus C, Perneczky R, Jahn H, Fiszer M, Maler J M, Zimmermann R, Bruckmoser R, Kornhuber J, Lewczuk P. Amyloid beta peptide ratio 42/40 but not A beta 42 correlates with phospho-Tau in patients with low- and high-CSF A beta 40 load. J Neurochem. 2007 May; 101(4):1053-9
Zhong Z, Ewers M, Teipel S, Bürger K, Wallin A, Blennow K, He P, McAllister C, Hampel H, Shen Y. Levels of beta-secretase (BACE1) in cerebrospinal fluid as a predictor of risk in mild cognitive impairment. Arch Gen Psychiatry. 2007 June; 64(6):718-26
(2) Buerger K, Otto M, Teipel S J, Zinkowski R, Blennow K, DeBernardis J, Kerkman D, Schröder J, Schönknecht P, Cepek L, McCulloch C, Möller H J, Wiltfang J, Kretzschmar H, Hampel H. Dissociation between CSF total tau and tau protein phosphorylated at threonine 231 in Creutzfeldt-Jakob disease. Neurobiol Aging. 2006 January; 27(1):10-5
Bibl M, Mollenhauer B, Esselmann H, Schneider M, Lewczuk P, Welge V, Gross M, Falkai P, Kornhuber J, Wiltfang J. Cerebrospinal fluid neurochemical phenotypes in vascular dementias: original data and mini-review. Dement Geriatr Cogn Disord. 2008; 25(3):256-65
McRae A, Martins R N, Fonte J, Kraftsik R, Hirt L, Miklossy J. Cerebrospinal fluid antimicroglial antibodies in Alzheimer disease: a putative marker of an ongoing inflammatory process. Exp Gerontol. 2007 April; 42(4):355-63
Jellinger K A, Janetzky B, Attems J, Kienzl E. Biomarkers for early diagnosis of Alzheimer disease: ‘ALZheimer ASsociated gene’—a new blood biomarker? J Cell Mol Med. 2008 August; 12(4):1094-117
Finehout E J, Franck Z, Choe L H, Relkin N, Lee K H. Cerebrospinal fluid proteomic biomarkers for Alzheimer's disease. Ann Neurol. 2007 February; 61(2):120-9
Castaño E M, Roher A E, Esh C L, Kokjohn T A, Beach T. Comparative proteomics of cerebrospinal fluid in neuropathologically-confirmed Alzheimer's disease and non-demented elderly subjects. Neurol Res. 2006 March; 28(2):155-63
Zhang J, Goodlett D R, Quinn J F, Peskind E, Kaye J A, Zhou Y, Pan C, Yi E, Eng J, Wang Q, Aebersold R H, Montine T J. Quantitative proteomics of cerebrospinal fluid from patients with Alzheimer disease. J Alzheimers Dis. 2005 April; 7(2):125-33
(1) Simonsen A H, McGuire J, Hansson O, Zetterberg H, Podust V N, Davies H A, Waldemar G, Minthon L, Blennow K. Novel panel of cerebrospinal fluid biomarkers for the prediction of progression to Alzheimer dementia in patients with mild cognitive impairment. Arch Neurol. 2007 March; 64(3):366-70
Lescuyer P, Allard L, Zimmermann-Ivol C G, Burgess J A, Hughes-Frutiger S, Burkhard P R, Sanchez J C, Hochstrasser D F. Identification of post-mortem cerebrospinal fluid proteins as potential biomarkers of ischemia and neurodegeneration. Proteomics. 2004 August; 4(8):2234-41
Abdi F, Quinn J F, Jankovic J, McIntosh M, Leverenz J B, Peskind E, Nixon R, Nutt J, Chung K, Zabetian C, Samii A, Lin M, Hattan S, Pan C, Wang Y, Jin J, Zhu D, Li G J, Liu Y, Waichunas D, Montine T J, Zhang J. Detection of biomarkers with a multiplex quantitative proteomic platform in cerebrospinal fluid of patients with neurodegenerative disorders. J Alzheimers Dis. 2006 August; 9(3):293-348
Irani D N, Anderson C, Gundry R, Cotter R, Moore S, Kerr D A, McArthur J C, Sacktor N, Pardo C A, Jones M, Calabresi P A, Nath A. Cleavage of cystatin C in the cerebrospinal fluid of patients with multiple sclerosis. Ann Neurol. 2006 February; 59(2):237-47
(2) Hansson S F, Simonsen A H, Zetterberg H, Andersen O, Haghighi S, Fagerberg I, Andréasson U, Westman-Brinkmalm A, Wallin A, Rüetschi U, Blennow K. Cystatin C in cerebrospinal fluid and multiple sclerosis. Ann Neurol. 2007 Aug.; 62(2):193-6
Ravaglia G, Ford P, Maioli F, Chiappelli M, Montesi F, Tumini E, Mariani E, Licastro F, Patterson C. Blood inflammatory markers and risk of dementia: The Conselice Study of Brain Aging. Neurobiol Aging. 2007 December; 28(12):1810-20
Engelhart M J, Geerlings M I, Meijer J, Kiliaan A, Ruitenberg A, van Swieten J C, Stijnen T, Hofman A, Witteman J C, Breteler M M. Inflammatory proteins in plasma and the risk of dementia: the rotterdam study. Arch Neurol. 2004 May; 61(5):668-72
Motta M, Imbesi R, Di Rosa M, Stivala F, Malaguarnera L. Altered plasma cytokine levels in Alzheimer's disease: correlation with the disease progression. Immunol Lett. 2007 Nov. 30; 114(1):46-51
German D C, Gurnani P, Nandi A, Garner H R, Fisher W, Diaz-Arrastia R, O'Suilleabhain P, Rosenblatt K P. Serum biomarkers for Alzheimer's disease: proteomic discovery. Biomed Pharmacother. 2007 August; 61(7):383-9
Ray S, Britschgi M, Herbert C, Takeda-Uchimura Y, Boxer A, Blennow K, Friedman L F, Galasko D R, Jutel M, Karydas A, Kaye J A, Leszek J, Miller B L, Minthon L, Quinn J F, Rabinovici G D, Robinson W H, Sabbagh M N, So Y T, Sparks D L, Tabaton M, Tinklenberg J, Yesavage J A, Tibshirani R, Wyss-Coray T. Classification and prediction of clinical Alzheimer's diagnosis based on plasma signaling proteins. Nat Med. 2007 November; 13(11):1359-62
Graff-Radford N R, Crook J E, Lucas J, Boeve B F, Knopman D S, Ivnik R J, Smith G E, Younkin L H, Petersen R C, Younkin S G. Association of low plasma Abeta42/Abeta40 ratios with increased imminent risk for mild cognitive impairment and Alzheimer disease. Arch Neurol. 2007 March; 64(3):354-62
van Oijen M, Hofman A, Soares H D, Koudstaal P J, Breteler M M. Plasma Abeta (1-40) and Abeta (1-42) and the risk of dementia: a prospective case-cohort study. Lancet Neurol. 2006 August; 5(8):655-60
Sundelöf J, Giedraitis V, Irizarry M C, Sundström J, Ingelsson E, Rönnemaa E, Arnlöv J, Gunnarsson M D, Hyman B T, Basun H, Ingelsson M, Lannfelt L, Kilander L. Plasma beta amyloid and the risk of Alzheimer disease and dementia in elderly men: a prospective, population-based cohort study. Arch Neurol. 2008 February; 65(2): 256-63
Song M S, Mook-Jung I, Lee H J, Min J Y, Park M H. Serum anti-amyloid-beta antibodies and Alzheimer's disease in elderly Korean patients. J Int Med Res. 2007 May-Jun.; 35(3):301-6
Pesaresi M, Lovati C, Bertora P, Mailland E, Galimberti D, Scarpini E, Quadri P, Forloni G, Mariani C. Plasma levels of beta-amyloid (1-42) in Alzheimer's disease and mild cognitive impairment. Neurobiol Aging. 2006 June; 27(6):904-5
Fukumoto H, Tennis M, Locascio J J, Hyman B T, Growdon J H, Irizarry M C. Age but not diagnosis is the main predictor of plasma amyloid beta-protein levels. Arch Neurol. 2003 July; 60(7):958-64
Kosaka T, Imagawa M, Seki K, Arai H, Sasaki H, Tsuji S, Asami-Odaka A, Fukushima T, Imai K, Iwatsubo T. The beta APP717 Alzheimer mutation increases the percentage of plasma amyloid-beta protein ending at A beta42 (43). Neurology. 1997 March; 48(3):741-5
Scheuner D, Eckman C, Jensen M, Song X, Citron M, Suzuki N, Bird T D, Hardy J, Hutton M, Kukull W, Larson E, Levy-Lahad E, Viitanen M, Peskind E, Poorkaj P, Schellenberg G, Tanzi R, Wasco W, Lannfelt L, Selkoe D, Younkin S. Secreted amyloid beta-protein similar to that in the senile plaques of Alzheimer's disease is increased in vivo by the presenilin 1 and 2 and APP mutations linked to familial Alzheimer's disease. Nat Med. 1996 August; 2(8):864-70
Sobów T, Flirski M, Kloszewska I, Liberski P P. Plasma levels of alpha beta peptides are altered in amnestic mild cognitive impairment but not in sporadic Alzheimer's disease. Acta Neurobiol Exp (Wars). 2005; 65(2):117-24
Tamaoka A, Fukushima T, Sawamura N, Ishikawa K, Oguni E, Komatsuzaki Y, Shoji S. Amyloid beta protein in plasma from patients with sporadic Alzheimer's disease. J Neurol Sci. 1996 Sep. 15; 141 (1-2):65-8
Vanderstichele H, Van Kerschaver E, Hesse C, Davidsson P, Buyse M A, Andreasen N, Minthon L, Wallin A, Blennow K, Vanmechelen E. Standardization of measurement of beta-amyloid (1-42) in cerebrospinal fluid and plasma. Amyloid. 2000 December; 7(4):245-58
Abdullah L, Paris D, Luis C, Quadros A, Parrish J, Valdes L, Keegan A P, Mathura V, Crawford F, Mullan M. The influence of diagnosis, intra- and inter-person variability on serum and plasma Abeta levels. Neurosci Lett. 2007 Nov. 27; 428 (2-3):53-8
Freeman S H, Raju S, Hyman B T, Frosch M P, Irizarry M C. Plasma Abeta levels do not reflect brain Abeta levels. J Neuropathol Exp Neurol. 2007 April; 66(4):264-71
Mehta P D, Pirttila T, Patrick B A, Barshatzky M, Mehta S P. Amyloid beta protein 1-40 and 1-42 levels in matched cerebrospinal fluid and plasma from patients with Alzheimer disease. Neurosci Lett. 2001 May 18; 304 (1-2):102-6
Giedraitis V, Sundelöf J, Irizarry M C, Gårevik N, Hyman B T, Wahlund L O, Ingelsson M, Lannfelt L. The normal equilibrium between CSF and plasma amyloid beta levels is disrupted in Alzheimer's disease. Neurosci Lett. 2007 Nov. 12; 427(3):127-31
Blasko I, Jellinger K, Kemmler G, Krampla W, Jungwirth S, Wichart I, Tragl K H, Fischer P. Conversion from cognitive health to mild cognitive impairment and Alzheimer's disease: prediction by plasma amyloid beta 42, medial temporal lobe atrophy and homocysteine. Neurobiol Aging. 2008 January; 29(1):1-11
Mehta P D, Pirttilä T, Mehta S P, Sersen E A, Aisen P S, Wisniewski H M. Plasma and cerebrospinal fluid levels of amyloid beta proteins 1-40 and 1-42 in Alzheimer disease. Arch Neurol. 2000 January; 57(1):100-5
Brettschneider S, Morgenthaler N G, Teipel S J, Fischer-Schulz C, Bürger K, Dodel R, Du Y, Möller H J, Bergmann A, Hampel H. Decreased serum amyloid beta (1-42) autoantibody levels in Alzheimer's disease, determined by a newly developed immuno-precipitation assay with radiolabeled amyloid beta (1-42) peptide. Biol Psychiatry. 2005 Apr. 1; 57(7):813-6
Bibl M, Esselmann H, Mollenhauer B, Weniger G, Welge V, Liess M, Lewczuk P, Otto M, Schulz J B, Trenkwalder C, Kornhuber J, Wiltfang J. Blood-based neurochemical diagnosis of vascular dementia: a pilot study. J. Neurochem. 2007 October; 103(2):467-74
Walsh D M, Selkoe D J. A beta oligomers—a decade of discovery. J. Neurochem. 2007 June; 101(5): 1172-84
Lambert M P, Barlow A K, Chromy B A, Edwards C, Freed R, Liosatos M, Morgan T E, Rozovsky I, Trommer B, Viola K L, Wals P, Zhang C, Finch C E, Krafft G A, Klein W L. Diffusible, nonfibrillar ligands derived from Abeta 1-42 are potent central nervous system neurotoxins. Proc Natl Acad Sci USA. 1998 May 26; 95(11):6448-53.
Walsh D M, Klyubin I, Fadeeva J V, Cullen W K, Anwyl R, Wolfe M S, Rowan M J, Selkoe D J. Naturally secreted oligomers of amyloid beta protein potently inhibit hippocampal long-term potentiation in vivo. Nature. 2002 Apr. 4; 416(6880):535-9.
Kayed R, Sokolov Y, Edmonds B, McIntire T M, Milton S C, Hall J E, Glabe C G. Permeabilization of lipid bilayers is a common conformation-dependent activity of soluble amyloid oligomers in protein misfolding diseases. J Biol Chem. 2004 Nov. 5; 279(45):46363-6
Cleary J P, Walsh D M, Hofmeister J J, Shankar G M, Kuskowski M A, Selkoe D J, Ashe K H. Natural oligomers of the amyloid-beta protein specifically disrupt cognitive function. Nat Neurosci. 2005 January; 8(1):79-84.
Pitschke M, Prior R, Haupt M, Riesner D. Detection of single amyloid beta-protein aggregates in the cerebrospinal fluid of Alzheimer's patients by fluorescence correlation spectroscopy. Nat Med. 1998 July; 4(7):832-4
Santos A N, Torkler S, Nowak D, Schlittig C, Goerdes M, Lauber T, Trischmann L, Schaupp M, Penz M, Tiller F W, Böhm G. Detection of amyloid-beta oligomers in human cerebrospinal fluid by flow cytometry and fluorescence resonance energy transfer. J Alzheimers Dis. 2007 March; 11(1):117-25
Klyubin I, Betts V, Wetzel A T, Blennow K, Zetterberg H, Wallin A, Lemere C A, Cullen W K, Peng Y, Wisniewski T, Selkoe D J, Anwyl R, Walsh D M, Rowan M J. Amyloid beta protein dimer-containing human CSF disrupts synaptic plasticity: prevention by systemic passive immunization. J Neurosci. 2008 Apr. 16; 28(16):4231-7
Roher A E, Baudry J, Chaney M O, Kuo Y M, Stine W B, Emmerling M R. Oligomerization and fibril assembly of the amyloid-beta protein. Biochim Biophys Acta. 2000 Jul. 26; 1502(1):31-43
Stenh C, Englund H, Lord A, Johansson A S, Almeida C G, Gellerfors P, Greengard P, Gouras G K, Lannfelt L, Nilsson L N. Amyloid-beta oligomers are inefficiently measured by enzyme-linked immunosorbent assay. Ann Neurol. 2005 July; 58(1):147-50.
Englund H, Sehlin D, Johansson A S, Nilsson L N, Gellerfors P, Paulie S, Lannfelt L, Pettersson F E. Sensitive ELISA detection of amyloid-beta protofibrils in biological samples. J Neurochem. 2007 October; 103(1):334-45.
Schupf N, Tang M X, Fukuyama H, Manly J, Andrews H, Mehta P, Ravetch J, Mayeux R. Peripheral Abeta subspecies as risk biomarkers of Alzheimer's disease. Proc Natl Acad Sci USA. 2008 Sep. 16; 105(37):14052-7
Englund H, Degerman Gunnarsson M, Brundin R M, Hedlund M, Kilander L, Lannfelt L, Pettersson F E. Oligomerization partially explains the lowering of Abeta42 in Alzheimer's disease cerebrospinal fluid. Neurodegener Dis. 2009; 6(4):139-47. Epub 2009 Jun. 12
Hansson O, Zetterberg H, Vanmechelen E, Vanderstichele H, Andreasson U, Londos E, Wallin A, Minthon L, Blennow K. Evaluation of plasma Abeta(40) and Abeta(42) as predictors of conversion to Alzheimer's disease in patients with mild cognitive impairment. Neurobiol Aging. 2008 May 16. [Epub ahead of print]
Kessler J, Calabrese P, Kalbe E, Berger F. Ein neues Screening-Verfahren zur Unterstützung der Demenzdiagnostik. Psycho 26 343-347 (2000)
Folstein M F, Folstein S E, McHugh P R. “Mini-mental state”. A practical method for grading the cognitive state of patients for the clinician. J Psychiatr Res. 1975 November; 12(3): 189-98
Shulman K I, Shedletsky R, Silver I L. The challenge of time: Clock-drawing and cognitive function in the elderly. Int J Geriatr Psychiatry 1, 135-140 (1986)
Irie K, Murakami K, Masuda Y, Morimoto A, Ohigashi H, Ohashi R, Takegoshi K, Nagao M, Shimizu T, Shirasawa T. Structure of beta-amyloid fibrils and its relevance to their neurotoxicity: implications for the pathogenesis of Alzheimer's disease. J Biosci Bioeng. 2005 May; 99(5):437-47
(2) Simonsen A H, Hansson S F, Ruetschi U, McGuire J, Podust V N, Davies H A, Mehta P, Waldemar G, Zetterberg H, Andreasen N, Wallin A, Blennow K. Amyloid beta 1-40 quantification in CSF: comparison between chromatographic and immunochemical methods. Dement Geriatr Cogn Disord. 2007; 23(4):246-50
Casas C, Sergeant N, Itier J M, Blanchard V, Wirths O, van der Kolk N, Vingtdeux V, van de Steeg E, Ret G, Canton T, Drobecq H, Clark A, Bonici B, Delacourte A, Benavides J, Schmitz C, Tremp G, Bayer T A, Benoit P, Pradier L. Massive CA1/2 neuronal loss with intraneuronal and N-terminal truncated Abeta42 accumulation in a novel Alzheimer transgenic model. Am J. Pathol. 2004 October; 165(4):1289-300

Gelfanova V, Higgs R E, Dean R A, Holtzman D M, Farlow M R, Siemers E R, Boodhoo A, Qian Y W, He X, Jin Z, Fisher D L, Cox K L, Hale J E. Quantitative analysis of amyloid-beta peptides in cerebrospinal fluid using immunoprecipitation and MALDI-T of mass spectrometry. Brief Funct Genomic Proteomic. 2007 June; 6(2): 149-58.
Sergeant N, Bombois S, Ghestem A, Drobecq H, Kostanjevecki V, Missiaen C, Wattez A, David J P, Vanmechelen E, Sergheraert C, Delacourte A. Truncated beta-amyloid peptide species in pre-clinical Alzheimer's disease as new targets for the vaccination approach.
J Neurochem. 2003 June; 85(6): 1581-91
Xia W, Yang T, Shankar G, Smith I M, Shen Y, Walsh D M, Selkoe D J. A specific enzyme-linked immunosorbent assay for measuring beta-amyloid protein oligomers in human plasma and brain tissue of patients with Alzheimer disease. Arch Neurol. 2009 February; 66(2):190-9.
El-Agnaf O M, Mahil D S, Patel B P, Austen B M. Oligomerization and toxicity of beta-amyloid-42 implicated in Alzheimer's disease. Biochem Biophys Res Commun. 2000 Jul. 14; 273(3):1003-7
Howlett D R, Perry A E, Godfrey F, Swatton J E, Jennings K H, Spitzfaden C, Wadsworth H, Wood S J, Markwell R E. Inhibition of fibril formation in beta-amyloid peptide by a novel series of benzofurans. Biochem J. 1999 May 15; 340 (Pt 1):283-9

What is claimed is:

1. A method of diagnosing or monitoring a neurodegenerative disorder comprising:
(a) determining a first concentration (cₐ) of a target amyloid β (Abeta or Aβ) peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ less than 1.5 is indicative of a positive diagnosis for a neurodegenerative disorder.

2. The method according to claim 1, wherein the disaggregation step (b) comprises the use of: an alkali; a suitable solvent; or an alkali and a suitable solvent.

3. The method according to claim 2, wherein:
the alkali comprises sodium hydroxide or 500 mM sodium hydroxide; or
the suitable solvent comprises methanol or 50% (v/v) methanol.

4. The method according to claim 1, wherein the disaggregation step (b) comprises an incubation step.

5. The method according to claim 4, wherein the disaggregation step (b) comprises an incubation step at room temperature for at least 2 minutes or at least 10 minutes.

6. The method according to claim 1, wherein a ratio of c_{d}/cₐ less than 1.4, less than 1.3, less than 1.2, or less than 1.1 is indicative of a positive diagnosis for a neurodegenerative disorder.

7. The method according to claim 1, wherein
the target Aβ peptide comprises Aβ (x-y) including functional equivalents thereof;
x is defined as an integer selected from 1, 2, 3, 4, 5, 6, 7, 8, 9, 10 and 11; and
y is defined as an integer selected from 38, 39, 40, 41, 42 and 43.

8. The method according to claim 7, wherein
x is an integer selected from 1, 2, 3 and 11; or
y is an integer selected from 38, 40 or 42.

9. The method according to claim 1, wherein the target Aβ peptide is selected from the group consisting of SEQ ID NOs. 1 to 24, including functional equivalents thereof.

10. The method according to claim 1, wherein the target Aβ peptide comprises:
Aβ (1-42) of SEQ ID NO: 1;
Aβ (1-40) of SEQ ID NO: 2;
Aβ (1-40) of SEQ ID NO. 2 and Aβ (1-42) of SEQ ID NO. 1;
Aβ (3-38) of SEQ ID NO. 13; or
Aβ (11-38) of SEQ ID NO. 19,
including functional equivalents thereof.

11. The method according to claim 9, wherein
the target Aβ peptide is selected from the group consisting of SEQ ID NOs. 13 to 24, including functional equivalents thereof; and
the glutamate residue at the N-terminus of said target Aβ peptide is cyclized to pyroglutamate.

12. The method according to claim 9, wherein
the target Aβ peptide is selected from the group consisting of SEQ ID NOs. 1 to 6, including functional equivalents thereof; and
the aspartate residues at amino acid positions 1, 7, or 1 and 7 are converted to isoasparate.

13. The method according to claim 9, wherein
the target Aβ peptide is selected from the group consisting of SEQ ID NOs. 7 to 12, including functional equivalents thereof; and
the aspartate residue at amino acid position 6 is converted to isoasparate.

14. The method according to claim 9, wherein
the target Aβ peptide is selected from the group consisting of SEQ ID NOs. 13 to 18, including functional equivalents thereof; and
the aspartate residue at amino acid position 5 is converted to isoasparate.

15. The method according to claim 1, wherein the biological sample comprises one or more of blood, serum, urine, cerebrospinal fluid (CSF), plasma, lymph, saliva, sweat, pleural fluid, synovial fluid, tear fluid, bile or pancreas secretion.

16. The method according to claim 15, wherein the biological sample is plasma.

17. The method according to claim 1, wherein determining the concentration of the target Aβ peptide comprises:
i) contacting the biological sample with at least two different capture antibodies;
ii) detecting a resulting immune complex;
iii) destroying the immune complex; and
iv) quantifying the captured Aβ peptides.

18. The method according to claim 17, wherein the at least two different capture antibodies are each specific for a different epitope on the target Aβ peptide.

19. The method according to claim 17, wherein the capture antibodies are selected from the group consisting of:
3D6, Epitope 1-5;
pAb-EL16, Epitope 1-7;
2H4, Epitope 1-8;
1E11, Epitope 1-8;
20.1, Epitope 1-10;
Rabbit Anti-Aβ Polyclonal Antibody, Epitope 1-14;
AB10, Epitope 1-16;
82E1, Epitope 1-16;
pAb1-42, Epitope 1-11;
NAB228, Epitope 1-11;
DE2, Epitope 1-16;
DE2B4, Epitope 1-17;
6E10, Epitope 1-17;
10D5, Epitope 3-7;
WO-2, Epitope 4-10;
1A3, Epitope 5-9;
pAb-EL21, Epitope 5-11;
310-03, Epitope 5-16;
Chicken Anti-Human Aβ Polyclonal Antibody, Epitope 12-28;
Chicken Anti-Human Aβ Polyclonal Antibody, Epitope 25-35;
Rabbit Anti-Human Aβ Polyclonal Antibody, Epitope N-terminal;
Rabbit Anti-Human Aβ Polyclonal Antibody;
12C3, Epitope 10-16;
16C9, Epitope 10-16;
19B8, Epitope 9-10;
pAb-EL26, Epitope 11-26;
BAM90.1, Epitope 13-28;
Rabbit Anti-beta-Amyloid (pan) Polyclonal Antibody, Epitope 15-30;
22D12, Epitope 18-21;
266, Epitope 16-24;
pAb-EL17, Epitope 15-24;
4G8, Epitope 17-24;
Rabbit Anti-Aβ Polyclonal Antibody, Epitope 22-35;
G2-10, Epitope 31-40;
Rabbit Anti-Aβ, aa 32-40 Polyclonal Antibody;
EP1876Y, Epitope x-40;
G2-11, Epitope 33-42;
16C11, Epitope 33-42;
21F12, Epitope 34-42;
1A10, Epitope 35-40;
D-17 Goat anti-Aβ antibody, Epitope C-terminal,
pyro-Glu Abeta antibodies, Aβ 5-5-6, Aβ 6-1-6, Aβ 17-4-3, Aβ 24-2-3;
Pyro-Glu Abeta monoclonal mouse antibody clone 2-48;
Pyro-Glu Abeta rabbit polyclonal antibody;
Pyro-Glu Abeta monoclonal mouse antibody clone 8E1;
Anti-Human Amyloidβ (N3pE) Rabbit rabbit polyclonal IgG;
Anti-Human Aβ N3pE (8E1) monoclonal mouse IgG Fab; and
isoAsp antibody.

20. The method according to claim 17, wherein the capture antibodies are selected from the group consisting of 3D6, BAN50, 82E1, 6E10, WO-2, 266, BAM90.1, 4G8, G2-10, 1A10, BA27, 11A5-B10, 12F4, and 21F12.

21. The method according to claim 17, wherein the at least two different capture antibodies comprise a pair selected from the group consisting of:
4G8 and 11A5-B10;
3D6 and 4G8;
6E10 and 4G8;
82E1 and 4G8;
4G8 and 12F4;
4G8 and 21F12;
3D6 and 21F12;
6E10 and 21F12;
BAN50 and 4G8;
3D6 and 11A5-B10;
3D6 and 1A10;
3D6 and BA27;
6E10 and 11A5-B10;
6E10 and 1A10;
6E10 and BA27;
4G8 and 11A5-B10;
4G8 and 1A10;
4G8 and BA27;
4G8 and 12F4; and
4G8 and 21F12.

22. The method according to claim 17, wherein the detection of the complex comprises reacting a secondary antibody with each capture antibody.

23. The method according to claim 22, wherein the secondary antibodies comprise anti-mouse antibodies or anti-rabbit antibodies.

24. The method according to claim 22, wherein the secondary antibodies comprise labeled secondary antibodies.

25. The method according to 17, wherein
the secondary antibodies are immobilized on magnetic beads; and
the magnetic beads carrying the immune complex are separated from the biological sample using a magnetic separator.

26. The method according to claim 17, wherein the destruction of the immune complex is performed in the presence of 50% (v/v) Methanol/0.5% (v/v) formic acid.

27. The method according to claim 17, wherein the detected immune complex is quantified.

28. The method according to claim 17, wherein the captured Aβ peptides are quantified by a sandwich ELISA; Amyloid β 1-40 HTRF Assay; Alphascreen Assay, Multiplex Assay Systems, mass spectrometry; or Western Blot analysis.

29. The method according to claim 28, wherein the captured Aβ peptides are quantified by a sandwich ELISA.

30. The method according to claim 29, wherein the sandwich ELISA comprises:
a first antibody, which is specific for the complete N-terminus of Aβ (x-y);
a detection antibody, which is specific for the C-terminus ending at amino acid y of Aβ (x-y);
x is defined as an integer selected from 1, 2, 3, 4, 5, 6, 7, 8, 9, 10 and 11; and
y is defined as an integer selected from 38, 39, 40, 41, 42 and 43.

31. The method according to claim 30, wherein the sandwich ELISA comprises:
(i) a first antibody, which is specific for the complete N-terminus of Aβ (1-42) of SEQ ID NO. 1; and a detection antibody, which is specific for the C-terminus ending at amino acid 42 of Aβ (1-42) of SEQ ID NO. 1;
(ii) a first antibody, which is specific for the C-terminus of Aβ (1-42) of SEQ ID NO. 1; and a detection antibody, which is specific for the complete N-terminus starting with Asp-Ala-Glu of Aβ (1-42) of SEQ ID NO. 1;
(iii) a first antibody, which is specific for the complete N-terminus of Aβ (1-40) of SEQ ID NO. 2; and a detection antibody, which is specific for the C-terminus ending at amino acid 40 of Aβ (1-40) of SEQ ID NO. 2;
(iv) a first antibody, which is specific for the C-terminus of Aβ (1-40) of SEQ ID NO. 2; and a detection antibody, which is specific for the complete N-terminus starting with Asp-Ala-Glu of Aβ (1-40) of SEQ ID NO. 2;

32. The method according to claim 29, wherein the sandwich ELISA comprises:
a first antibody, which is specific for the complete N-terminus of an Aβ target peptide selected from the group consisting of SEQ ID NOs. 13 to 24; and
a detection antibody, which is specific for the C-terminus of said Aβ target peptide selected from the group consisting of SEQ ID NOs. 13 to 24.

33. The method according to claim 32, wherein the sandwich ELISA comprises:
(i) a first antibody, which is specific for the complete N-terminus of Aβ (3-38) of SEQ ID NO. 13; and a detection antibody, which is specific for the C-terminus ending at amino acid 38 of Aβ (3-38) of SEQ ID NO. 13; or
(ii) a first antibody, which is specific for the complete N-terminus of Aβ (11-38) of SEQ ID NO. 19; and a detection antibody, which is specific for the C-terminus ending at amino acid 38 of Aβ (11-38) of SEQ ID NO. 19.

34. The method according to 32, wherein
the N-terminus of the Aβ target peptide selected from the group consisting of SEQ ID NOs. 13 to 24 is cyclized to pyroglutamate; and
the first antibody is specifically detecting the pyroglutamated form of said Aβ target peptide selected from the group consisting of SEQ ID NOs. 13 to 24

35. The method according to claim 29, wherein
the first antibody is immobilized;
the detection antibody is labeled; or
the first antibody is immobilized and the detection antibody is labeled.

36. The method according to claim 30, wherein an ELISA-Kit for the quantification of Aβ (x-y) is used.

37. The method according to claim 36, wherein the ELISA-Kit quantifies Aβ (1-40) of SEQ ID NO, 2 or Aβ (1-42) of SEQ ID NO. 1.

38. The method of claim 1, wherein
the ratio of c_{d}/cₐ is determined for at least two biological samples; and
the at least two biological samples are taken on different occasions from a test subject.

39. The method of claim 38 comprising comparing the c_{d}/cₐ ratios of the at least two biological samples.

40. The method according to claim 1, wherein the neurodegenerative disorder comprises Alzheimer's disease.

41. The method according to claim 40, wherein a ratio of c_{d}/cₐ less than 1.5 is indicative of a differential diagnosis for Alzheimer's disease.

42. The method according to claim 1, wherein the neurodegenerative disorder comprises early stage Alzheimer's disease and a ratio of c_{d}/cₐ less than 1.5 is indicative of a differential diagnosis for early stage Alzheimer's disease.

43. The method according to claim 1, wherein the neurodegenerative disorder comprises Mild Cognitive Impairment.

44. The method according to claim 42, wherein the early stage Alzheimer's disease comprises Mild Cognitive Impairment.

45. A method of determining an oligomeric state of a target amyloid β (Abeta or Aβ) peptide in a biological sample comprising:
(a) determining a first concentration (cₐ) of a target Aβ peptide in a biological sample;
(b) disaggregating the target Aβ peptide from step (a);
(c) determining a second concentration (c_{d}) of the disaggregated Aβ peptide; and
(d) determining the ratio of c_{d}/cₐ, wherein the value of the second concentration (c_{d}) is divided by the value of the first concentration cₐ;
wherein a ratio of c_{d}/cₐ which is greater than 1 is indicative of the presence of oligomeric Aβ.

46. The method according to claim 43, wherein the disaggregation step (b) comprises the use of an alkali.

47. A method of monitoring efficacy of a therapy in a subject having, suspected of having, or of being predisposed to, a neurodegenerative disorder comprising determining an oligomeric state of a target amyloid β (Abeta or Aβ) peptide according to claim 45 in a biological sample from a test subject.

48. The method of claim 47, wherein
comparing the c_{d}/cₐ ratios of at least two biological samples taken on different occasions from the subject.

49. A kit for diagnosing a neurodegenerative disorder comprising a suitable alkali and instructions to use said kit in accordance with the methods according to claim 1.


SEQUENCE LISTING (24 sequences)

SEQ ID NO: 1 - protein, 42 aa, Homo sapiens [sequence omitted]

SEQ ID NO: 2 - protein, 40 aa, Homo sapiens
DAEFRHDSGYEVHHQKLVFFAEDVGSNKGAIIGLMVGGVV

SEQ ID NO: 3 - protein, 38 aa, Homo sapiens
DAEFRHDSGYEVHHQKLVFFAEDVGSNKGAIIGLMVGG

SEQ ID NO: 4 - protein, 39 aa, Homo sapiens
DAEFRHDSGYEVHHQKLVFFAEDVGSNKGAIIGLMVGGV

SEQ ID NO: 5 - protein, 41 aa, Homo sapiens
DAEFRHDSGYEVHHQKLVFFAEDVGSNKGAIIGLMVGGVVI

SEQ ID NO: 6 - protein, 43 aa, Homo sapiens
DAEFRHDSGYEVHHQKLVFFAEDVGSNKGAIIGLMVGGVVIAT

SEQ ID NO: 7 - protein, 37 aa, Homo sapiens
AEFRHDSGYEVHHQKLVFFAEDVGSNKGAIIGLMVGG

SEQ ID NO: 8 - protein, 38 aa, Homo sapiens
AEFRHDSGYEVHHQKLVFFAEDVGSNKGAIIGLMVGGV

SEQ ID NO: 9 - protein, 39 aa, Homo sapiens
AEFRHDSGYEVHHQKLVFFAEDVGSNKGAIIGLMVGGVV

SEQ ID NO: 10 - protein, 40 aa, Homo sapiens
AEFRHDSGYEVHHQKLVFFAEDVGSNKGAIIGLMVGGVVI

SEQ ID NO: 11 - protein, 41 aa, Homo sapiens
AEFRHDSGYEVHHQKLVFFAEDVGSNKGAIIGLMVGGVVIA

SEQ ID NO: 12 - protein, 42 aa, Homo sapiens
AEFRHDSGYEVHHQKLVFFAEDVGSNKGAIIGLMVGGVVIAT

SEQ ID NO: 13 - protein, 36 aa, Homo sapiens
EFRHDSGYEVHHQKLVFFAEDVGSNKGAIIGLMVGG

SEQ ID NO: 14 - protein, 37 aa, Homo sapiens
EFRHDSGYEVHHQKLVFFAEDVGSNKGAIIGLMVGGV

SEQ ID NO: 15 - protein, 38 aa, Homo sapiens
EFRHDSGYEVHHQKLVFFAEDVGSNKGAIIGLMVGGVV

SEQ ID NO: 16 - protein, 39 aa, Homo sapiens
EFRHDSGYEVHHQKLVFFAEDVGSNKGAIIGLMVGGVVI

SEQ ID NO: 17 - protein, 40 aa, Homo sapiens
EFRHDSGYEVHHQKLVFFAEDVGSNKGAIIGLMVGGVVIA

SEQ ID NO: 18 - protein, 41 aa, Homo sapiens
EFRHDSGYEVHHQKLVFFAEDVGSNKGAIIGLMVGGVVIAT

SEQ ID NO: 19 - protein, 28 aa, Homo sapiens
EVHHQKLVFFAEDVGSNKGAIIGLMVGG

SEQ ID NO: 20 - protein, 29 aa, Homo sapiens
EVHHQKLVFFAEDVGSNKGAIIGLMVGGV

SEQ ID NO: 21 - protein, 30 aa, Homo sapiens
EVHHQKLVFFAEDVGSNKGAIIGLMVGGVV

SEQ ID NO: 22 - protein, 31 aa, Homo sapiens
EVHHQKLVFFAEDVGSNKGAIIGLMVGGVVI

SEQ ID NO: 23 - protein, 32 aa, Homo sapiens
EVHHQKLVFFAEDVGSNKGAIIGLMVGGVVIA

SEQ ID NO: 24 - protein, 33 aa, Homo sapiens
EVHHQKLVFFAEDVGSNKGAIIGLMVGGVVIAT